(19) United States
(12) Patent Application Publication
(10) Pub. No.: US 2026/0157872 A1
(43) Pub. Date: Jun. 11, 2026

(54) ADJUSTABLE DEVICE AND METHOD TO ADJUST ADJUSTABLE DEVICE FOR IMMOBILIZING AND STABILIZING LIMBS

(71) Applicants: Shilpa Pravin Karle, Pune (IN); Ashish Pravin Karle, Pune (IN)

(72) Inventors: Shilpa Pravin Karle, Pune (IN); Ashish Pravin Karle, Pune (IN); Prashant Ramesh Madake, Pune (IN); Sanket Balasaheb Dukare, Ahmednagar (IN)

(73) Assignees: Shilpa Pravin Karle, Pune (IN); Ashish Pravin Karle, Pune (IN)

(21) Appl. No.: 19/387,688

(22) Filed: Nov. 13, 2025

(30) Foreign Application Priority Data
Dec. 6, 2024 (IN) 202421096341

(51) Int. Cl. A61F 5/058; A61F 5/01

(52) U.S. Cl. CPC A61F 5/05841; A61F 2005/0158; A61F 2005/0167; A61F 2005/0172

(57) ABSTRACT

Adjustable-device (100) and method to adjust the adjustable-device (100) for immobilizing and stabilizing limbs eliminates a conventional fixed rigid structure by providing linear and rotational adjustability, before and after application, allowing it to adapt to different limb sizes and changes in limb size due to swelling or its reduction. Adjustable-device (100) includes wearable-supports (10a, 10b, 10c, 10d, 10e), linear-adjustable-links (20a, 20b), and rotational-adjustable-joints (30a). The wearable-supports (10a, 10b, 10c, 10d, 10e) are adjustably-wearable on the limbs (05a, 05b, 05c, 05d, 05e) adjustable between an expanded-configuration and a gripping-configuration. The linear-adjustable-links (20a, 20b) are connected to the wearable-supports (10a, 10b, 10c) and are defined to linearly-adjust the distance between the wearable-supports (10a, 10b, 10c). The rotational-adjustable-joints (30a) are pivotally-connected to the wearable-supports (10a, 10b) and rotationally-adjusts the dimensional-variance of the adjacently-disposed wearable-supports (10a, 10b), the dimensional-variance is the difference-in-sizes of the adjacently-disposed wearable-supports (10a, 10b) worn on the limbs (05a, 05b).

### FIELD OF THE INVENTION

The present disclosure pertains to an adjustable device and a method for adjusting the adjustable device to immobilize and stabilize limbs, typically bones/tubular bones. Specifically, the present disclosure pertains to an adjustable device that is adjustably configured to fit on a body part of a user, and a method for adjusting the adjustable device to immobilize and stabilize tubular bones of a limb to facilitate fracture healing.

### BACKGROUND OF THE INVENTION

Immobilizing and stabilizing fractured limbs, especially bones, has traditionally involved various casting devices and methods like fiberglass casts or traditional plaster, namely Plaster of Paris (POP) (for example patent document WO2000035501-Casting material), UV-Activated Casts (for example patent document U.S. Pat. No. 3,881,473-Ultraviolet light curable orthopedic cast material and method of forming an orthopedic cast), 3D-Printed Casts (for example patent document US20170216078-3D printed splint and cast), Resin Sleeve Casts (Cast 21) and Thermoformable Casts (for example patent document EP0914358-Thermoformable cast poly [methyl methacrylate]). Despite advancements in materials and techniques, most casting solutions still face common disadvantages that impact patient comfort, adaptability, and ease of use.

Key Disadvantages of Existing Casting Solutions:
1. Non-Adjustability: Conventional casts, once applied, cannot be adjusted to accommodate changes in limb swelling or reduction in swelling. For example, if the limb swells, the cast becomes tight, causing discomfort or restricted blood circulation, often requiring the cast to be cut open to relieve pressure. As the swelling subsides, the cast may become loose, leading to inadequate support for proper healing. Since these casts cannot be readjusted, they often need to be replaced multiple times during the recovery period, significantly adding to medical costs. Additionally, casts such as UV-curable, resin sleeve, thermoformable, and 3D-printed casts require different sizes for individuals with varying limb dimensions, further complicating inventory management and increasing overall healthcare expenses.
2. Inflexibility of removal and reuse of casts: Conventional casts cannot be removed and reapplied easily without compromising structural integrity. In events where dressing changes are required near or around the fracture site—such as in open wounds, post-surgical care, or infections—the cast often needs to be cut and discarded. Reapplying a new cast after every intervention causes patient discomfort, increases treatment time, and adds to medical costs.
3. Dependency on hardening agents and specialized equipment: Many casting methods rely on specific hardening agents, such as water, UV light, heat, or resin, and require specialized equipment for application or removal. This dependency complicates application, limits their availability outside clinical settings, and can delay immediate casting in emergency situations.
4. Lack of provision of wrist angle: Conventional casts do not incorporate a prefabricated fixed wrist angle—typically around 20 degrees—which is considered ideal for optimal fracture healing. As a result, there may be a risk of improper positioning during application, which can compromise alignment and negatively affect the healing process.
5. Patient discomfort and hygiene issues: Traditional casts, especially those made of plaster and fiberglass, are not breathable, which causes sweat and moisture to accumulate inside. This trapped moisture can lead to skin irritation, itching, and unpleasant odors over time. Additionally, these casts are not waterproof, so any external exposure to water can further worsen skin conditions and compromise hygiene.
6. Time-consuming application process: Advanced casting methods like UV-activated, 3D-printed, and thermoformable casts are often time-intensive, either due to lengthy hardening processes, custom printing, or heating requirements. This can delay application, which is particularly problematic in situations requiring immediate immobilization.
7. Limited reusability and size constraints: Current casting options generally come in various fixed sizes, which reduces flexibility for different patient anatomies. Additionally, they are typically single-use for the same patient, which adds to healthcare costs and environmental waste, particularly with materials that cannot be easily recycled or reused.
8. Limited degree of freedom: Conventional casting options do not cater to adjustability for accommodating various limb sizes (diameter and length adjustments) along with rotational adjustments.
9. Limited shelf life: Conventional casts have a limited shelf life, after which they must be discarded if unused. This results in material wastage, increased inventory turnover, and higher operational costs for medical facilities.

The above limitations highlight the need for an adjustable device and a method that provides adjustability (linear and rotational), minimal dependency on hardening agents or specialized equipment, improved patient comfort, rapid application, and enhanced reusability across a range of limb sizes and types.

Hence, there is a need for an adjustable device and a method for adjusting the adjustable device to immobilize and stabilize limbs that can alleviate some of the above-mentioned drawbacks.

### OBJECT OF THE INVENTION

Some of the objects of the arrangement of the present disclosure are aimed at ameliorating one or more problems of the prior art or at least providing a useful alternative, and are listed herein below.
A principal object of the present disclosure is to provide an adjustable device and a method to adjust the adjustable device for immobilizing and stabilizing limbs that is fully adjustable, linearly and rotationally, before and after application, allowing it to adapt to different limb sizes and changes in limb size due to swelling or reduction in swelling, thereby enhancing patient comfort and stability throughout the healing process, and which allows air ventilation to maintain hygiene and avoid discomfort.
An object of the present disclosure is to provide an adjustable device and a method to adjust the adjustable device for immobilizing and stabilizing limbs that allows for easy removal and reapplication to facilitate multiple wound inspections and dressing changes.
Still another object of the present disclosure is to provide an adjustable device and a method to adjust the adjustable device for immobilizing and stabilizing limbs that has a provision of maintaining a prefabricated fixed wrist angle.
Another object of the present disclosure is to provide an adjustable device and a method to adjust the adjustable device for immobilizing and stabilizing limbs that eliminates the need for traditional hardening agents like water, UV light, reactive resin or heat and avoids associated equipment, thereby simplifying the application and removal process and enabling immediate use without dependence on specialized equipment.
Yet another object of the present disclosure is to provide an adjustable device and a method to adjust the adjustable device for immobilizing and stabilizing limbs that allows air circulation for enhanced breathability and moisture control, while being waterproof and washable, thereby minimizing the risk of skin irritation, itching, and odor typically associated with conventional casting methods.
Still another object of the present disclosure is to provide an adjustable device and a method to adjust the adjustable device for immobilizing and stabilizing limbs that reduces waste and lowers medical costs by enabling multiple uses for the same patient and has an unlimited shelf life.
Another object of the present disclosure is to provide an adjustable device and a method to adjust the adjustable device for immobilizing and stabilizing limbs that is adjustable to accommodate a broad range of limb sizes and anatomies without requiring different cast sizes, making it a more universally applicable and convenient option.

Other objects and advantages of the present disclosure will be more apparent from the following description when read in conjunction with the accompanying figures, which are not intended to limit the scope of the present disclosure.

### SUMMARY OF THE INVENTION

The present invention discloses an adjustable-device for immobilizing and stabilizing limbs that is adjustable, linearly and rotationally, before and after application, allowing adaptation for varying limb sizes and fluctuations caused by swelling or its reduction, thereby improving patient comfort and ensuring stable support throughout recovery while also allowing air circulation for better hygiene, preventing discomfort, and being both waterproof and washable. The adjustable-device includes wearable-supports, linear-adjustable-links, and rotational-adjustable-joints. The wearable-supports are adjustably-wearable on the limbs and are adjustable between an expanded-configuration and a gripping-configuration. The linear-adjustable-links are connected to the wearable-supports and are defined to linearly-adjust the distance between the wearable-supports. The rotational-adjustable-joints are pivotally-connected to the wearable-supports and are defined to rotationally-adjust the dimensional-variance of the adjacently-disposed wearable-supports, in which the dimensional-variance is the difference-in-sizes of the adjacently-disposed wearable-supports worn on the limbs.
In one embodiment, each of the wearable-supports is defined with housings, connecting-elements, and locking-elements. The housings are connected by the connecting-elements to form a wearable-structure. The connecting-elements are adjustably-movable in the housings. The locking-elements are defined to lock and unlock the connecting-elements with the housings, in which the locking-elements lock the connecting-elements with the housings, defining a locking-configuration, and the locking-elements un-lock the connecting-elements with the housings, defining an un-locking-configuration.
Each of the linear-adjustable-links is defined with a female-element, a male-element which is adjustably and telescopically movable in the female-element and is defined with male-interlocking-teeth, and a locking-member which is defined on the female-element and configured to: —engage with the male-interlocking-teeth defining a locking-configuration and —disengage with the male-interlocking-teeth defining an un-locking-configuration.
Typically, each of the rotational-adjustable-joints is defined with sockets which are defined on the adjacently-disposed wearable-supports, and threaded-rods, each threaded-rod having one-end which is defined with a ball fitted in the socket and a threaded-portion threadably fitted in an enclosure having internal thread-receptacles, in which rotation of the enclosure extends and retracts the threaded-rods, the balls pivotally-rotate in the sockets.
In an additional embodiment, the adjustable-device includes an angular-connector connected between the angularly-disposed wearable-supports and a spaced-connector connected between the wearable-supports.

The present disclosure also discloses a method to adjust an adjustable-device for immobilizing and stabilizing limbs, in accordance with one embodiment. The method includes:
providing the adjustable-device defined with wearable-supports, linear-adjustable-links, and rotational-adjustable-joints, the linear-adjustable-links connected to the wearable-supports, the rotational-adjustable-joints pivotally-connected to the wearable-supports;
adjusting the wearable-supports to an expanded-configuration;
inserting the limbs in the wearable-supports;
adjusting linearly the linear-adjustable-links to linearly-adjust the distance between the wearable-supports to position the wearable-supports on the limbs;
adjusting the wearable-supports to a gripping-configuration; and
adjusting the rotational-adjustable-joints to rotationally-adjust the dimensional-variance of the adjacently-disposed wearable-supports, wherein the dimensional-variance is the difference-in-sizes of the adjacently-disposed wearable-supports worn on the limbs.
In one embodiment, the method includes the step of adjusting the wearable-supports defined with housings, connecting-elements, and locking-elements, —in the expanded-configuration includes unlocking, to achieve an unlocking-configuration, the locking-elements to disengage the connecting-elements with the housings to enable outward-movement of the connecting-elements in the housings for expansion of the wearable-supports for inserting the limbs; and
in the gripping-configuration by inwardly moving the connecting-elements in the housings to grip with the limbs and locking, to achieve a locking-configuration, the locking-elements to engage the connecting-elements with the housings for the gripping-configuration.
Typically, the method includes disengaging and reengaging the connecting-elements and the housings from the connecting-elements and the housings.
In one embodiment, the step of adjusting linearly the linear-adjustable-links, defined with a female-element, a male-element with male-interlocking-teeth, and a locking-member defined on the female-element, includes unlocking the locking-member to telescopically-adjust the male-element in the female-element and locking the locking-member to engage with the male-interlocking-teeth and arrest the male-element with the female-element.
Additionally, the method includes the step of adjusting the rotational-adjustable-joints defined with sockets which are defined on the adjacently-disposed wearable-supports, threaded-rods with balls and threaded-portions, and an enclosure having internal thread-receptacles, includes rotating the enclosure for extending and retracting the threaded-rods, the balls pivotally-rotate in the sockets.

### BRIEF DESCRIPTION OF DRAWINGS

The present disclosure will now be described with the help of the accompanying drawings, in which:

FIG. 1 illustrates a schematic perspective view of an adjustable-device (100), in accordance with one embodiment of the present disclosure, for immobilizing and stabilizing limbs (05a, 05b, 05c, 05d, 05e) of a user (05). The adjustable-device (100) is defined with wearable-supports (10a, 10b, 10c, 10d, 10e), linear-adjustable-links (20a, 20b), and rotational-adjustable-joints (30a), and a belt (45) is also shown which supports the adjustable-device (100) around the user's neck;

FIG. 2 illustrates a schematic perspective view of an adjustable-device (100), in accordance with another embodiment of the present disclosure, for immobilizing and stabilizing limbs (05a, 05b, 05c) of a user (05), and is defined with wearable-supports (10a, 10b, 10c), linear-adjustable-links (20a, 20b), and rotational-adjustable-joints (30a), and a belt (45) for neck support;

FIG. 3 illustrates a schematic perspective representation of the adjustable-device (100) with the belt (45);

FIG. 4 illustrates a perspective view of the adjustable-device (100) of FIG. 1;

FIG. 5 illustrates a front view of the adjustable-device (100) of FIGS. 1 and 4;

FIG. 6 illustrates a perspective view of the adjustable-device (100) of FIGS. 2, and 3;

FIG. 7 illustrates a front view of the adjustable-device (100) of FIGS. 2, 3, and 6;

FIG. 8 illustrates a typical exploded view of the adjustable-device (100) of FIG. 2;

FIG. 9 illustrates a schematic perspective view of the wearable-support (10c);

FIG. 10 illustrates a schematic exploded view of the wearable-support (10c);

FIG. 11 illustrates a schematic perspective view of the wearable-support (10b) with an integrated socket (30i) of the rotational-adjustable-joints (30a);

FIG. 12 illustrates a schematic exploded view of the wearable-support (10b) with the integrated socket (30i) of the rotational-adjustable-joints (30a);

FIG. 13 illustrates a schematic exploded view of the socket (30i);

FIG. 14 illustrates a schematic perspective view of the wearable-supports (10b and 10c) connected by the linear-adjustable-links (20b);

FIG. 15 illustrates a schematic perspective view of the wearable-support (10a);

FIG. 16 illustrates an exploded view of the wearable-support (10a);

FIG. 17 illustrates a perspective view of the linear-adjustable-links (20b) pivotally connected to the wearable-support (10c);

FIG. 18 illustrates a perspective view of an alternative linear-adjustable-links (20a, 20b) defined with a female-element (20i), a male-element (20ii) with male-interlocking-teeth (20iii), a locking-member (20iv), a stopper (20v) with a slider-hole (20va), in which the female-element (20i) is in an extended configuration with the male-element (20ii);

FIG. 19 illustrates a typical perspective view of the linear-adjustable-links (20a, 20b) defined with the female-element (20i), the male-element (20i) with male-interlocking-teeth (20iii), and a locking-member (20iv) in a retracted configuration;

FIG. 20 illustrates a schematic exploded view of the linear-adjustable-links (20a, 20b);

FIG. 21 illustrates a schematic perspective view of a first locking configuration showing the housing (10i), the connecting-elements (10v, 10viii), and integrated-locking-elements (10ix);

FIG. 22 illustrates a schematic exploded view of the first locking-configuration of FIG. 21, showing the housing (10i), the connecting-elements (10v, 10viii), and locking-elements (10ix);

FIG. 23 illustrates a schematic perspective view of the typical housings (10i, 10ii, 10iii, 10iv);

FIG. 24 illustrates a schematic perspective view of the connecting-elements (10v, 10vi, 10vii, 10viii);

FIG. 25 illustrates another perspective view of the locking-elements (10ix);

FIG. 26 illustrates a schematic perspective view of the first locking-configuration of FIG. 21 in unlocked-state;

FIG. 27 illustrates a schematic top view of the first locking-configuration of FIG. 21 in unlocked-state;

FIG. 28 illustrates a schematic perspective view of the first locking-configuration of FIG. 21 in locked-state;

FIG. 29 illustrates a schematic top view of the first locking-configuration of FIG. 21 in locked-state;

FIG. 30 illustrates a schematic view of a second locking-configuration showing the housing (10i), the connecting-elements (10v, 10viii), and removably-connected locking-elements (10ix);

FIG. 31 illustrates an exploded view of the second locking-configuration of FIG. 30 showing the housing (10i), the connecting-elements (10v, 10viii), and the removably-connected locking-elements (10ix);

FIG. 32 illustrates a schematic view of a third locking-configuration showing the housing (10i), the connecting-elements (10v, 10viii), and pivotally-connected locking-elements (10ix) in an unlocked configuration;

FIG. 33 illustrates a schematic view of the third locking-configuration of FIG. 32 showing the housing (10i), the connecting-elements (10v, 10viii), and the pivotally-connected locking-elements (10ix) in locked configuration;

FIG. 34 illustrates a schematic view of a fourth locking-configuration showing the housing (10i), the connecting-elements (10v, 10viii), and bolt locking-elements (10ix);

FIG. 35 illustrates a schematic view of the fourth locking-configuration of FIG. 34 showing the housing (10i), the connecting-elements (10v, 10viii) and bolt locking-elements (10ix) enclosed in the housing (10i);

FIG. 36 illustrates a schematic view of a fifth locking-configuration showing the housing (10i) with openings (10ia), connecting-elements (10v, 10viii) with slide-rail (10x), and locking-elements (10ix);

FIG. 37 illustrates another schematic view of the fifth locking-configuration of FIG. 36;

FIG. 38 illustrates an exploded view of the fifth locking-configuration of FIG. 36;

FIG. 39 illustrates a perspective view of the locking-elements (10ix) of the fifth locking-configuration;

FIG. 40 illustrates a schematic perspective view of threaded-rods (30ii) with balls (30iii) and threaded-portions (30iv) threadably fitted in an enclosure (30v);

FIG. 41 illustrates a perspective view of an angular-connector (40) for connecting the wearable-supports (10c, 10d);

FIG. 42 illustrates a perspective view of spaced-connectors (41) connected between wearable-supports (10d, 10e);

FIG. 43 illustrates an exploded view of the wearable-supports (10d, 10e), the angular-connector (40), and the spaced-connectors (41);

FIG. 44 illustrates an assembled view of the wearable-supports (10d, 10e), the angular-connector (40), and the spaced-connectors (41);

FIG. 45 illustrates a perspective view of disengaging the connecting-elements (10v, 10viii) and the housing (10i) from the remaining connecting-elements (10vi, 10vii) and the housings (10ii, 10iii, 10iv) of wearable supports (10d, 10e), expanding the wearable-supports (10a, 10b, 10c, 10d, 10e) and inserting limbs (05a, 05b, 05c, 05d, 05e) in the housings (10i, 10ii, 10iii) of the wearable supports (10d, 10e);

FIG. 46 illustrates a perspective view of compressing the wearable-supports (10a, 10b, 10c);

FIG. 47 illustrates a perspective view of assembling the connecting-elements (10v, 10viii) with the housings (10ii, 10iv) of the wearable supports (10d, 10e);

FIG. 48 illustrates a perspective view of expanding the wearable-supports (10a, 10b, 10c);

FIG. 49 illustrates a perspective view of inserting the limbs (05a, 05b, 05c) in the expanded wearable-supports (10a, 10b, 10c);

FIG. 50 illustrates a perspective view of compressing the wearable-supports (10a, 10b, 10c);

FIG. 51 illustrates a front view of the adjustable-device (100) worn on limbs (05a, 05b, 05c);

FIG. 52 illustrates a perspective view of the linear-adjustable-links (20a) that is defined with an angular-adjustment-mechanism (20c) defined to angularly adjust the linear-adjustable-links (20a);

FIG. 53 illustrates an exploded view of the angular-adjustment-mechanism (20c) of FIG. 52;

FIG. 54 illustrates a perspective view of the angular-adjustment-mechanism (20c) with the linear-adjustable-link (20ai); and

FIG. 55 illustrates a perspective view of the linear-adjustable-link (20ai).

### DETAILED DESCRIPTION OF THE INVENTION

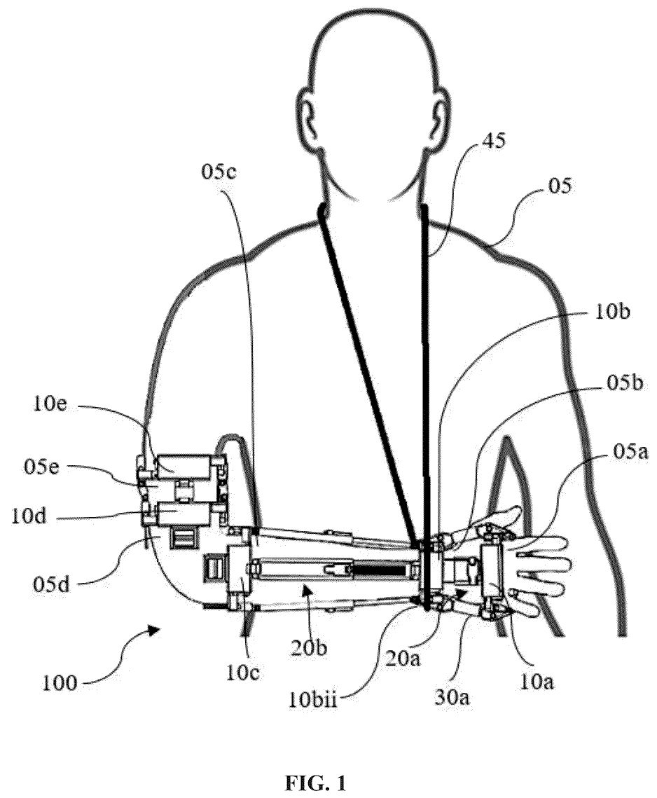

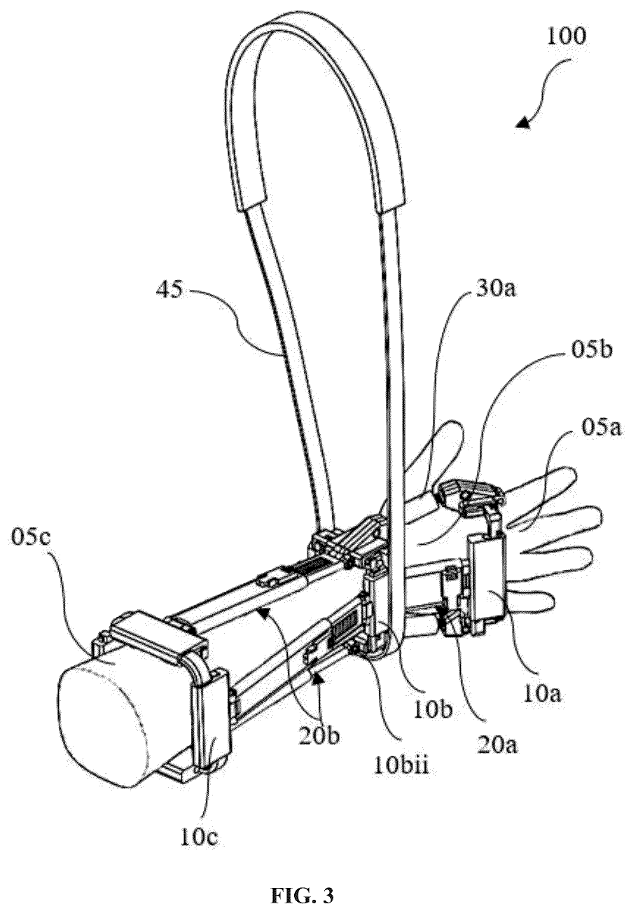

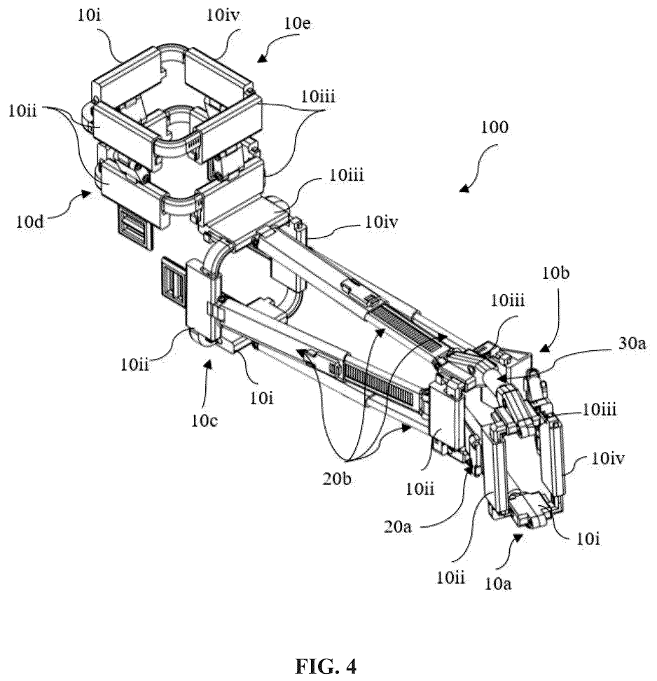

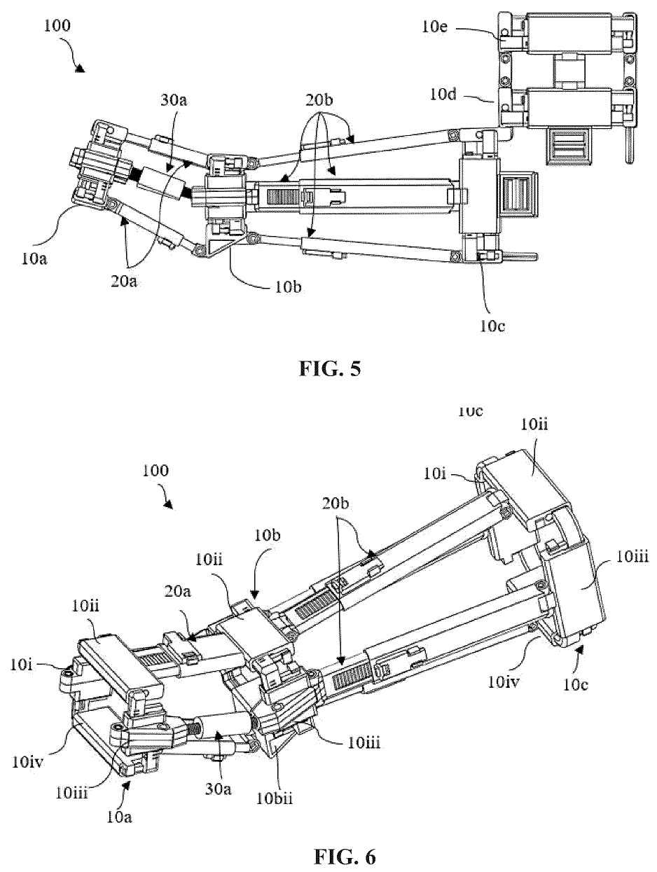

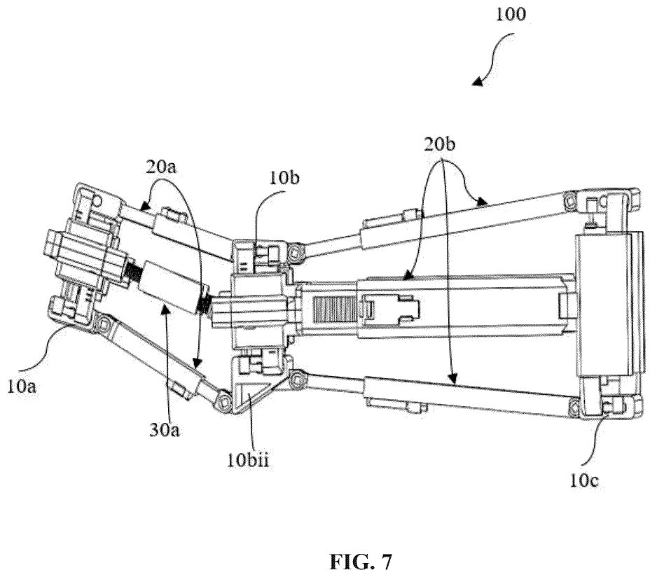

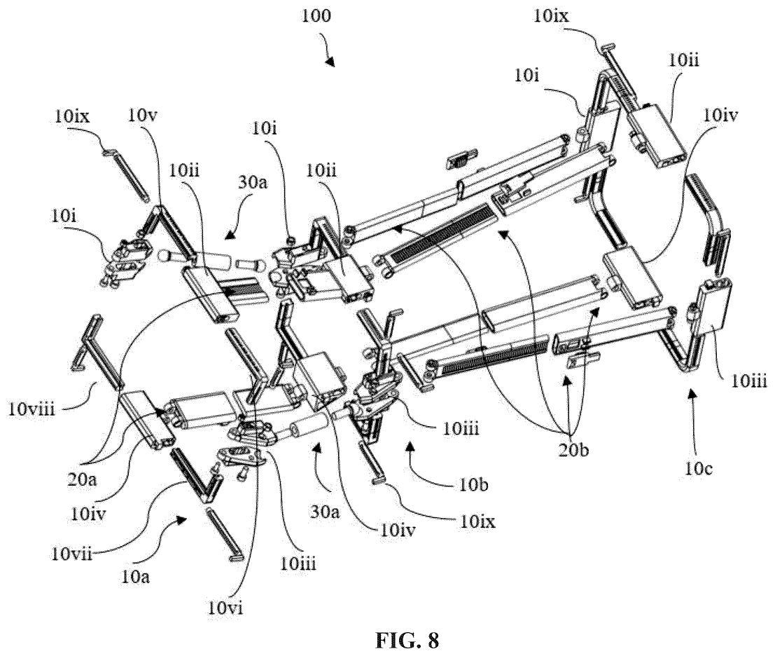

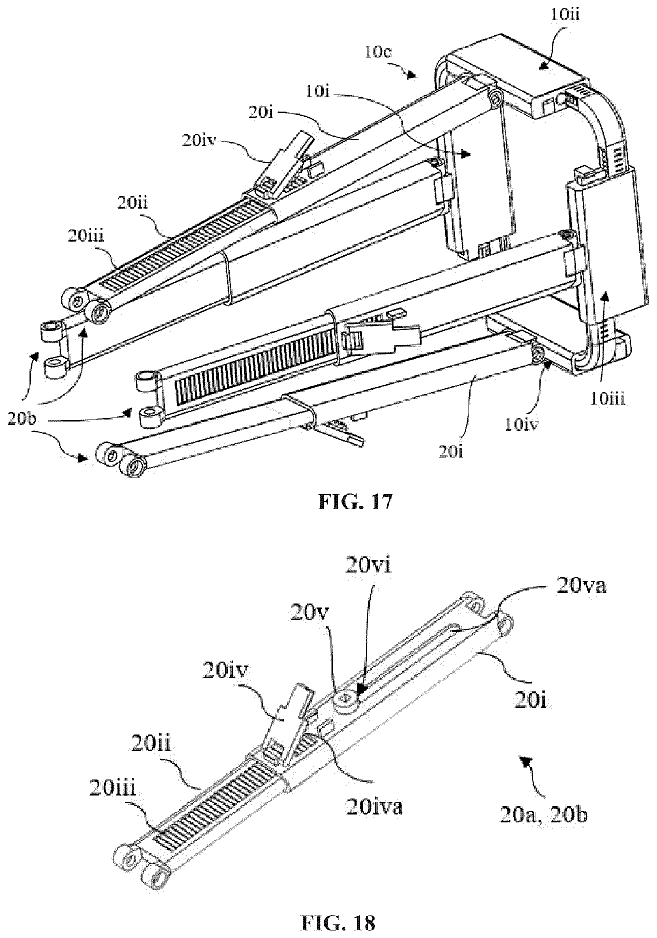

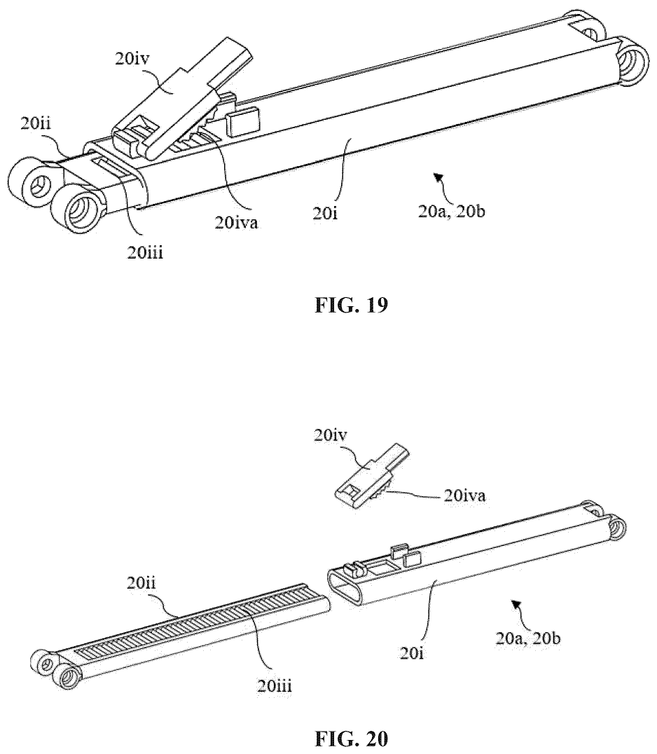

Referring now to the drawings, FIGS. 1 to 55, where the present invention is generally referred to with numeral (100), it can be observed that an adjustable-device (100), in accordance with an embodiment, for immobilizing and stabilizing limbs (05a, 05b, 05c, 05d, 05e) of a user (05) is provided which includes wearable-supports (10a, 10b, 10c, 10d, 10e), linear-adjustable-links (20a, 20b), and rotational-adjustable-joints (30a). The wearable-supports (10a, 10b, 10c, 10d, 10e) are adjustably-wearable on the limbs (05a, 05b, 05c, 05d, 05e) and are adjustable between an expanded-configuration and a gripping-configuration. Herein, the expanded-configuration is the state where the wearable-supports (10a, 10b, 10c, 10d, 10e) are widened or opened to allow easy insertion of the limbs (05a, 05b, 05c, 05d, 05e). Herein, the gripping-configuration is the state where the wearable-supports (10a, 10b, 10c, 10d, 10e) are compressed to securely hold and immobilize the limbs (05a, 05b, 05c, 05d, 05e). The linear-adjustable-links (20a, 20b) are connected (pivotally and/or fixedly) to the wearable-supports (10a, 10b, 10c) and are defined to linearly-adjust the distance between the wearable-supports (10a, 10b, 10c). The rotational-adjustable-joints (30a) are pivotally-connected to the wearable-supports (10a, 10b) and are defined to rotationally-adjust the dimensional-variance of the adjacently-disposed wearable-supports (10a, 10b), in which the dimensional-variance is the difference-in-sizes of the adjacently-disposed wearable-supports (10a, 10b) worn on the limbs (05a, 05b). The difference-in-sizes refers to the variation in dimensions—such as diameter, length, circumference, or perimeter—of the wearable-supports (10a, 10b) that corresponds to the anatomical differences of the adjacent limbs (05a, 05b) on which they are worn.

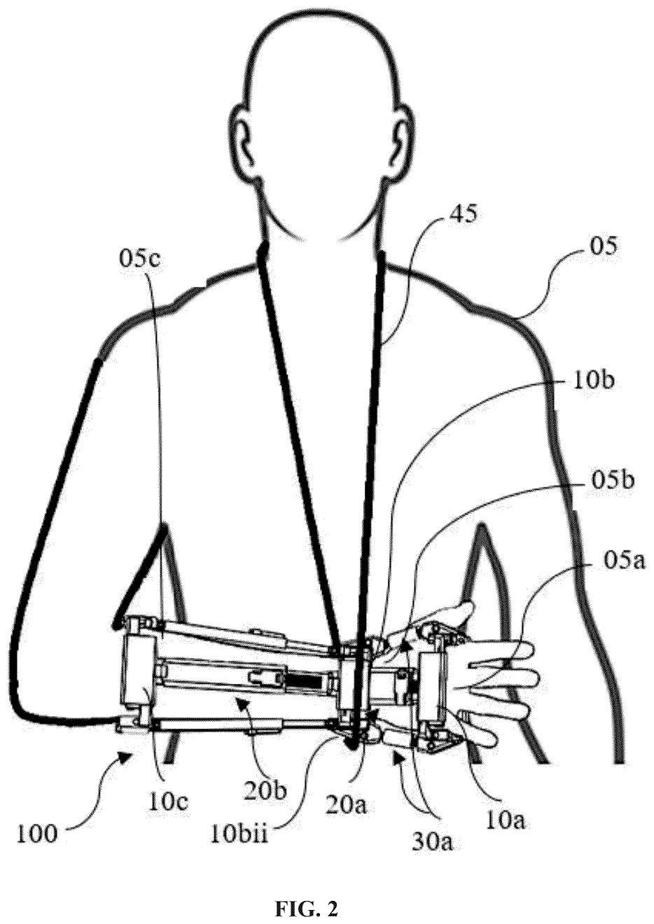

In accordance with one exemplary embodiment as illustrated in FIGS. 1, 4, and 5, the adjustable-device (100) includes the wearable-supports (for finger-side wrist portion 10a, for wrist and forearm 10b and 10c and for upper arm 10d and 10e) for supporting the limbs (05a, 05b, 05c, 05d, 05e). In another exemplary embodiment, as illustrated in FIGS. 2, 3, 6, 7, and 8, the adjustable-device (100) includes the wearable-supports (for finger-side wrist portion 10a and for wrist and forearm 10b and 10c) for supporting the limbs (05a, 05b, 05c).

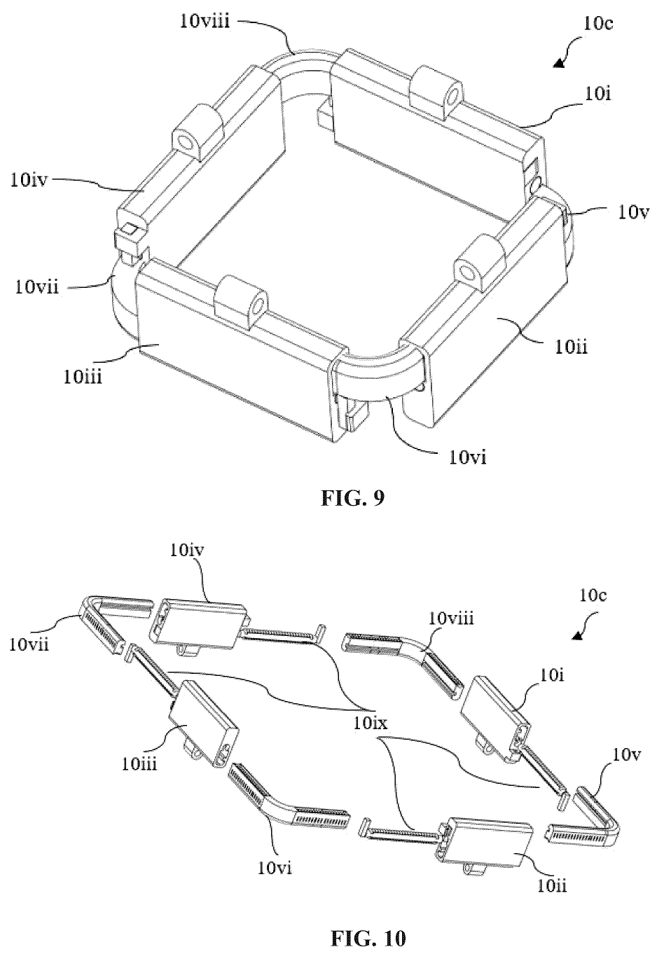

The wearable-supports (10a, 10b, 10c, 10d, 10e) are adjustably-wearable on the limbs (05a, 05b, 05c, 05d, 05e) and are manually adjustable. The wearable-supports (10a, 10b, 10c, 10d, 10e) are deployed between the expanded-configuration, for allowing the limbs (05a, 05b, 05c, 05d, 05e) to move in the adjustable-device (100), and the gripping-configuration for allowing the adjustable-device (100) to be gripped with the limbs (05a, 05b, 05c, 05d, 05e). In one embodiment, the wearable-support (10c) is illustrated in FIGS. 9 and 10. The wearable-support (10c) is defined with housings (10i, 10ii, 10iii, 10iv), connecting-elements (10v, 10vi, 10vii, 10viii), and locking-elements (10ix). The housings (10i, 10ii, 10iii, 10iv) are connected by the connecting-elements (10v, 10vi, 10vii, 10viii) to form a wearable-structure. The connecting-elements (10v, 10vi, 10vii, 10viii) are adjustably-movable in the housings (10i, 10ii, 10iii, 10iv). The locking-elements (10ix) are defined to lock and unlock the connecting-elements (10v, 10vi, 10vii, 10viii) with the housings (10i, 10ii, 10iii, 10iv). In one embodiment, the locking and unlocking is performed manually. The locking-elements (10ix) lock the connecting-elements (10v, 10vi, 10vii, 10viii) with the housings (10i, 10ii, 10iii, 10iv) defining a locking-configuration and the locking-elements (10ix) un-lock the connecting-elements (10v, 10vi, 10vii, 10viii) with the housings (10i, 10ii, 10iii, 10iv) defining an un-locking-configuration.

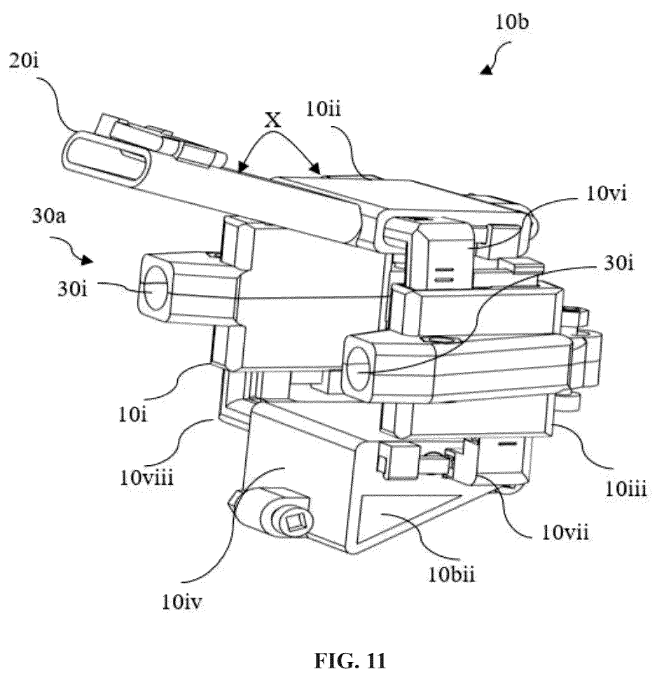

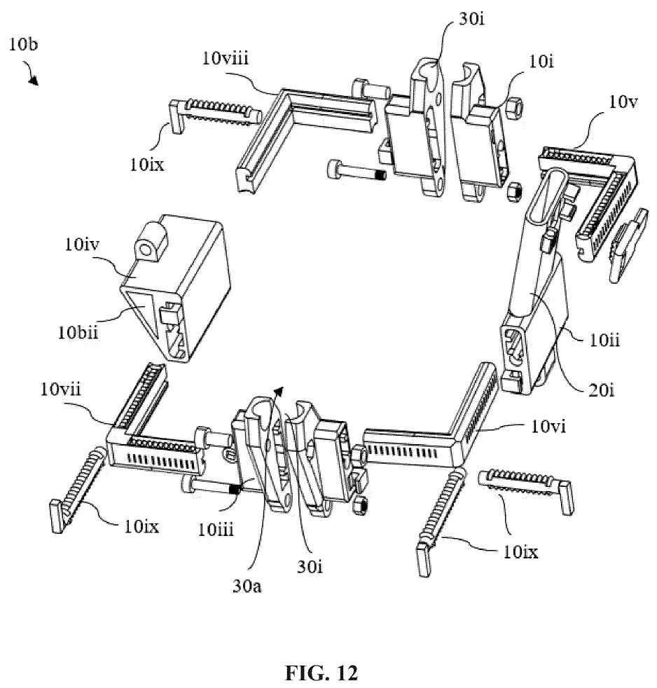

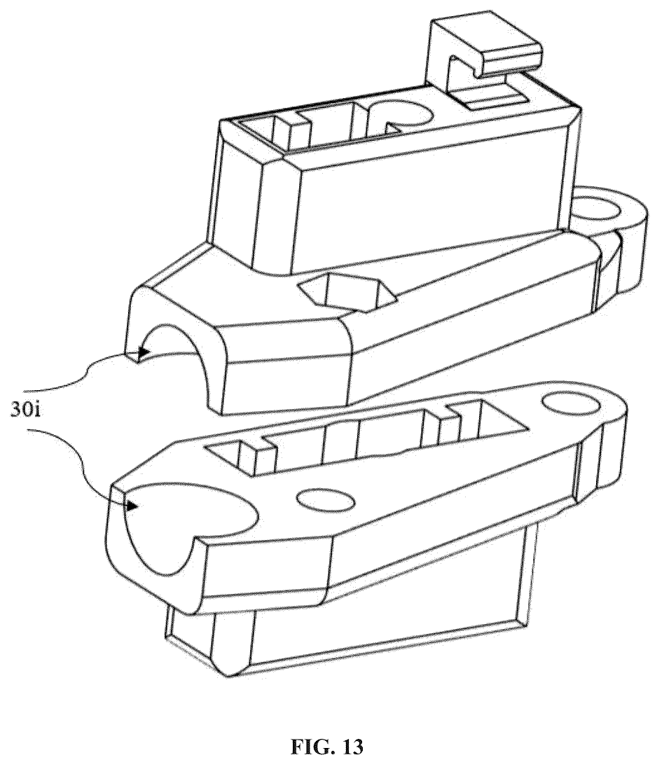

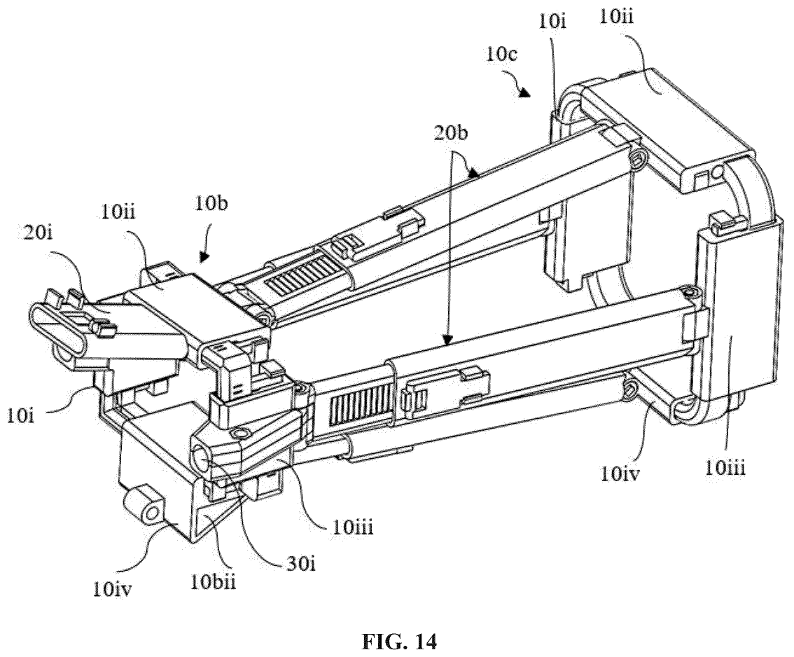

FIGS. 11 and 12 disclose the wearable-support (10b). The wearable-support (10b) is also defined with the housings (10i, 10ii, 10iii, 10iv), the connecting-elements (10v, 10vi, 10vii, 10viii), and the locking-elements (10ix) like the wearable-support (10c). Additionally, the wearable-support (10b) supports sockets (30i) of the rotational-adjustable-joints (30a). The exploded view of the sockets (30i) is illustrated in FIG. 13. Typically, the wearable-support (10b) includes an opening (10bii) for passing a belt (45), which is then worn around the neck of the user (05) for support as illustrated in FIGS. 1 and 2. The wearable-support (10b) is also connected to a female-element (20i) of the linear-adjustable-links (20a, 20b). In one embodiment, the female-element (20i) is defined at an angle in the range from 90 to 270 degrees with respect to the wearable-support (10b) and more preferably the angle (X-illustrated in FIG. 11) is in the range from 160 to 165 degrees so that the wrist extension angle recommended for healing, typically in the range from 15 to 20 degrees, is obtained. FIG. 14 illustrates the assembly of the wearable-support (10b) with the linear-adjustable-links (20b), which connect the wearable-support (10c).

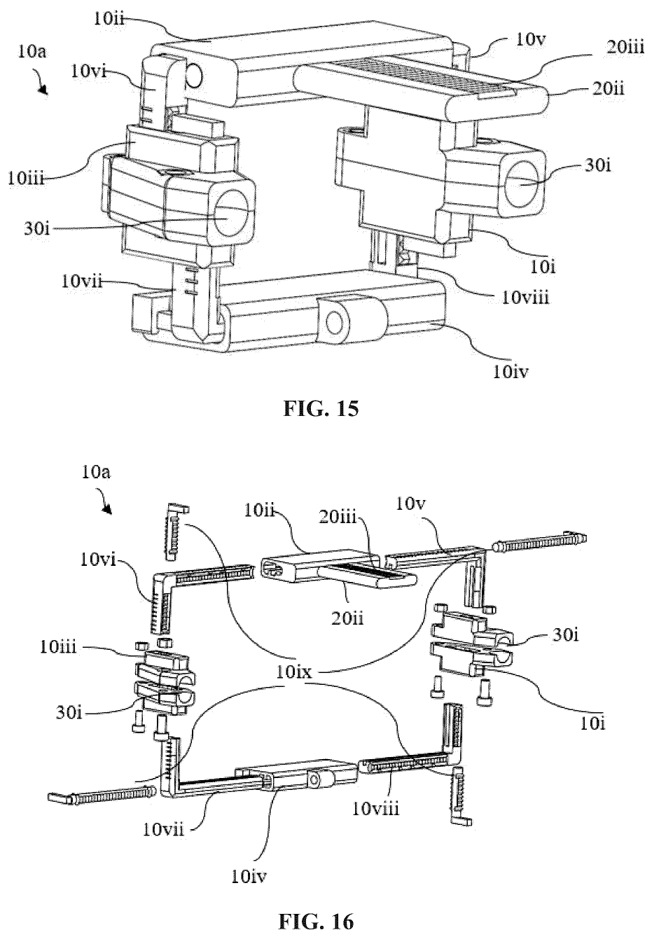

FIGS. 15 and 16 illustrate the wearable-support (10a) having the housings (10i, 10ii, 10iii, 10iv), the connecting-elements (10v, 10vi, 10vii, 10viii), and the locking-elements (10ix) like the wearable-supports (10b, 10c). Additionally, the wearable-support (10a) supports the sockets (30i) of the rotational-adjustable-joints (30a). The exploded view of the sockets (30i) is illustrated in FIG. 13. The wearable-support (10a) is also connected to a male-element (20ii) of the linear-adjustable-links (20a, 20b). The male-element (20ii) is inserted inside the female-element (20i) fitted with the wearable-support (10b).

In one embodiment, the wearable-supports (10a, 10b, 10c, 10d, 10e) can be of same sizes or the wearable-supports (10a, 10b, 10c, 10d, 10e) can be of different/variable sizes.

FIGS. 18 to 20 are the linear-adjustable-links (20a, 20b) that adjust (reduce or increase) the distance between the wearable-supports (10a, 10b, 10c). In one embodiment, the linear-adjustable-links (20b) are pivotally-connected to the wearable-supports (10b, 10c) as illustrated in FIG. 14. In one embodiment, the linear-adjustable-links (20a) are fixedly-connected to the wearable-supports (10a, 10b). In one embodiment, the linear-adjustable-links (20a, 20b) are defined with a female-element (20i), a male-element (20ii) with male-interlocking-teeth (20iii), and a locking-member (20iv). The male-element (20ii) is adjustably and telescopically movable in the female-element (20i) and is defined with the male-interlocking-teeth (20iii). The locking-member (20iv) is defined on the female-element (20i) and is defined with teeth (20iva). The teeth (20iva) engage with the male-interlocking-teeth (20iii), thus defining a locking-configuration. The teeth (20iva) disengage with the male-interlocking-teeth (20iii) defining an un-locking-configuration. In one embodiment, the adjustment of locking of the female-element (20i) and the male-element (20ii), unlocking of the female-element (20i) and the male-element (20ii), extending-out the male-element (20ii) from the female-element (20i), and inserting-in the male-element (20ii) into the female-element (20i) is performed manually. In an additional embodiment, as illustrated in FIG. 18, the linear-adjustable-links (20a, 20b) can be provided with a stopper (20v) introduced from the female-element (20i) that interacts with a corresponding hole (20vi) defined in the male-element (20ii) to prevent disengagement of the male-element (20ii) from the female-element (20i). A slider-hole (20va) is provided that provides a path for the stopper (20v). In an alternative embodiment, the linear-adjustable-links (20a, 20b) can be non-adjustable by providing different sizes (lengths) of linear-links.

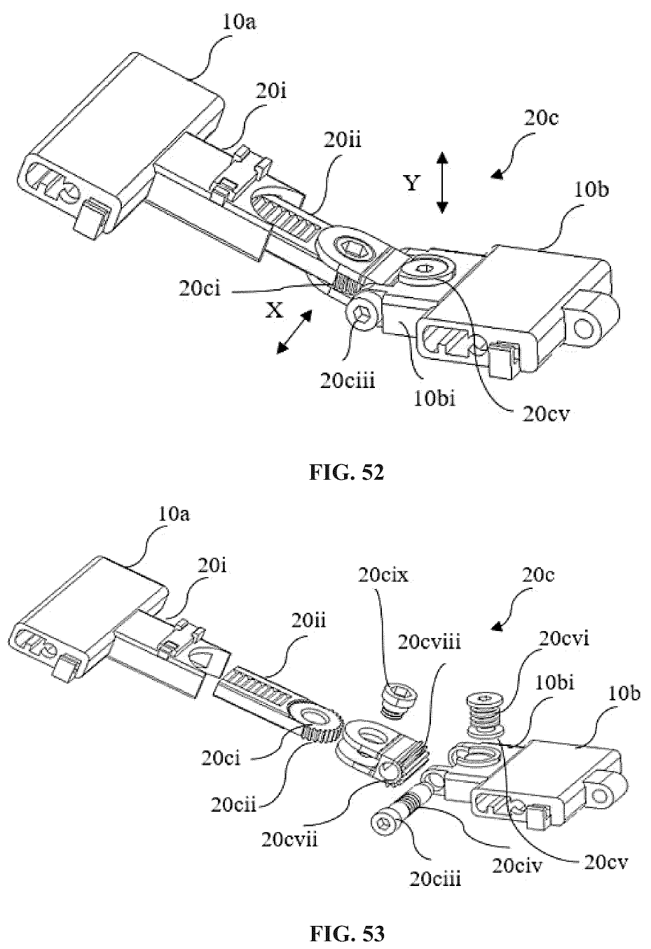

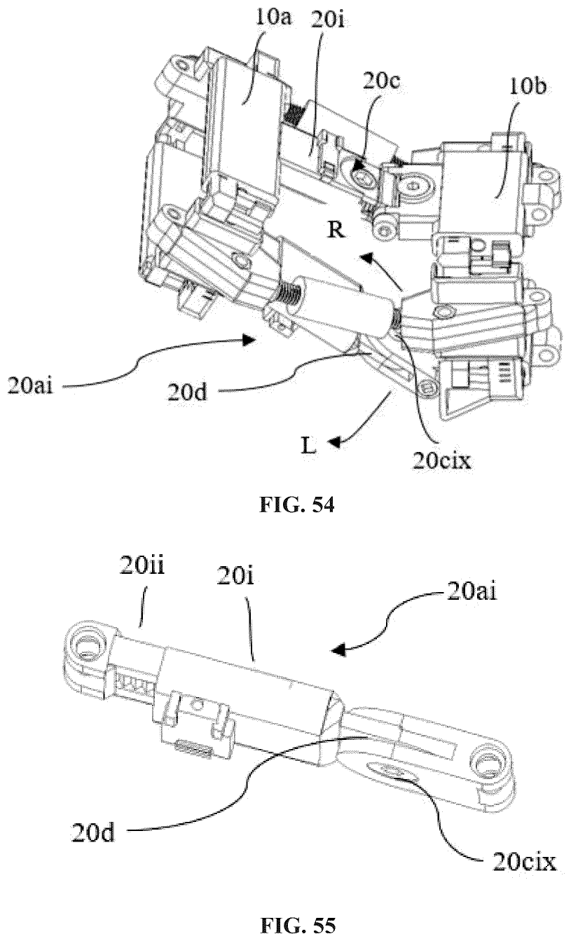

In an additional embodiment, as illustrated in FIGS. 52 to 55, the linear-adjustable-links (20a) are defined with an angular-adjustment-mechanism (20c) for angularly adjusting the linear-adjustable-links (20a). The angular-adjustment-mechanism (20c) is defined with a left-right-adjustment and a top-bottom adjustment. The left-right adjustment is movable in direction X, and the top-bottom adjustment is movable in direction Y, as illustrated in FIG. 52. The left-right-adjustment includes a worm-wheel (20ci) with teeth (20cii) and a teethed-worm (20ciii) with teeth (20civ). The teethed-worm (20ciii) is rotated to rotate the worm-wheel (20ci), enabling left and right movement depending upon clockwise and anticlockwise rotation of the teethed-worm (20ciii). The worm-wheel (20ci) is defined on the male-element (20ii). The teethed-worm (20ciii) is rotatably disposed in an extended-portion (10bi) of the wearable-support (10b). The teeth (20civ) are connected to the teeth (20cii). The top-bottom-adjustment includes a worm (20cv) with teeth (20cvi) and an angular-teethed-wheel (20cvii) with angular-teeth (20cviii). The worm (20cv) is disposed in the extended-portion (10bi) and has an axis transverse to the axis of the teethed-worm (20ciii). The angular-teethed-wheel (20cvii) extends to connect with the worm-wheel (20ci) by the use of a fastener (20cix). The teeth (20cvi) are connected to the angular-teeth (20cviii). The worm (20cv) is rotated to rotate the angular-teethed-wheel (20cvii) to achieve top-bottom adjustment (Y). In an additional embodiment, as illustrated in FIGS. 54 and 55, the linear-adjustable-link (20ai) is provided that has a freewheeling left-right movements, and the left-right movement of the linear-adjustable-link (20ai) is in accordance with the left-right adjustment of the linear-adjustable-link (20a). The linear-adjustable-link (20ai) differs from the linear-adjustable-link (20a) by using a toothless-wheel (20d) pivotally movable (in the left direction ‘L’ and in the right direction ‘R’) and is pivotally-connected by a fastener (20cix).

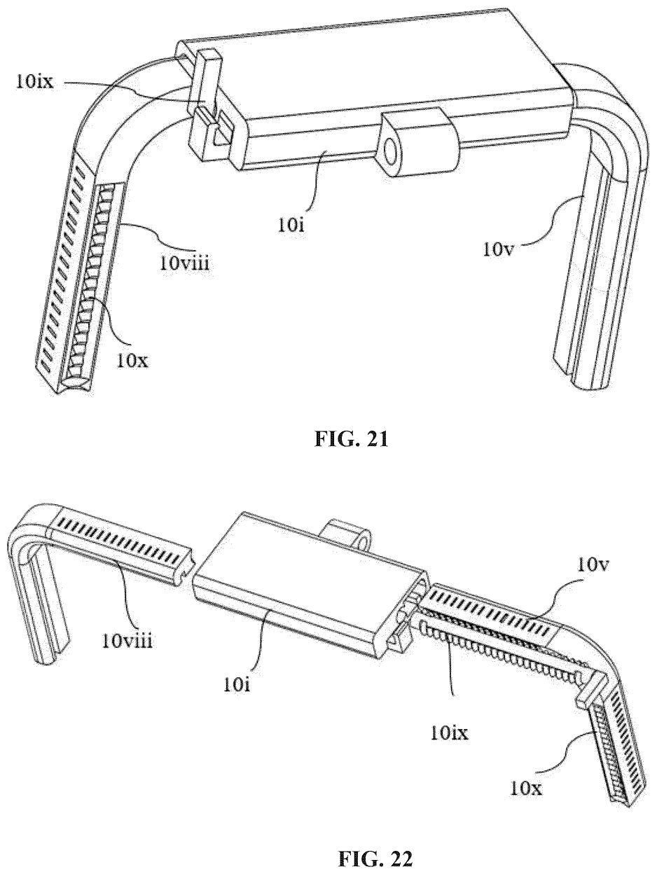

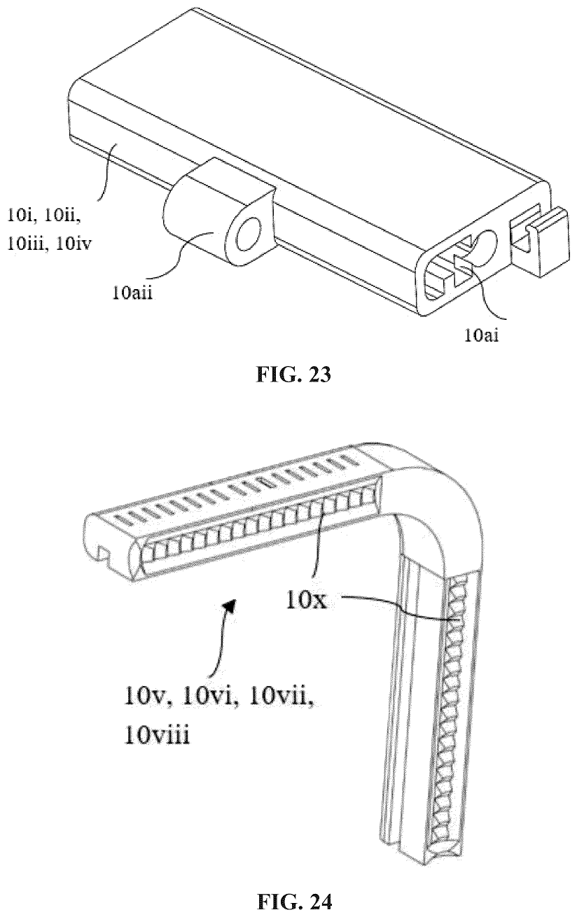

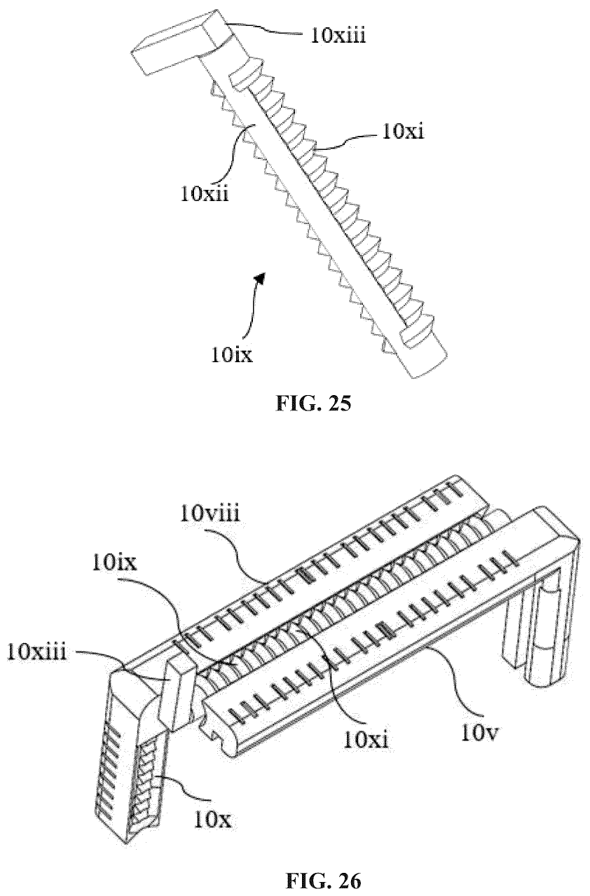

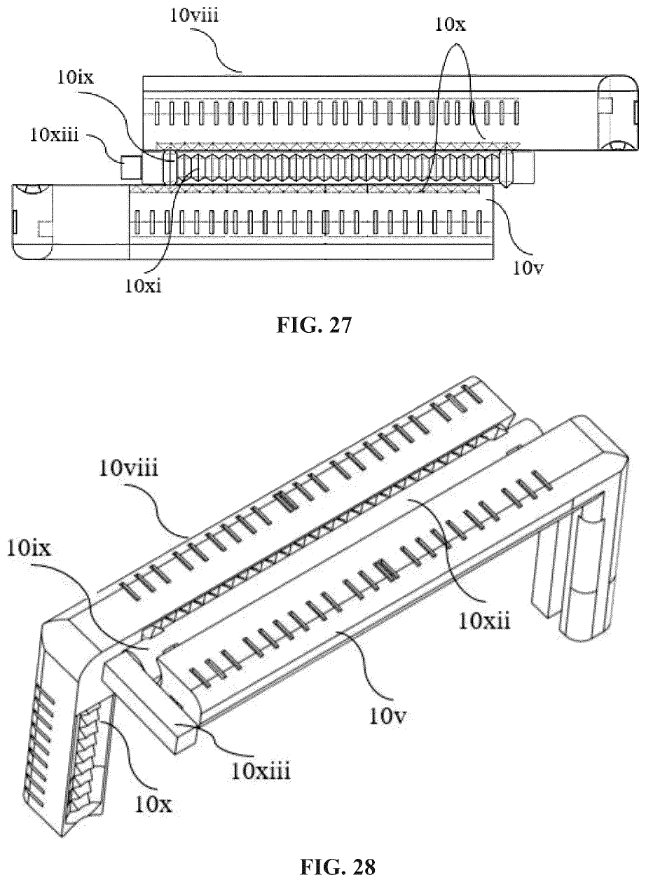

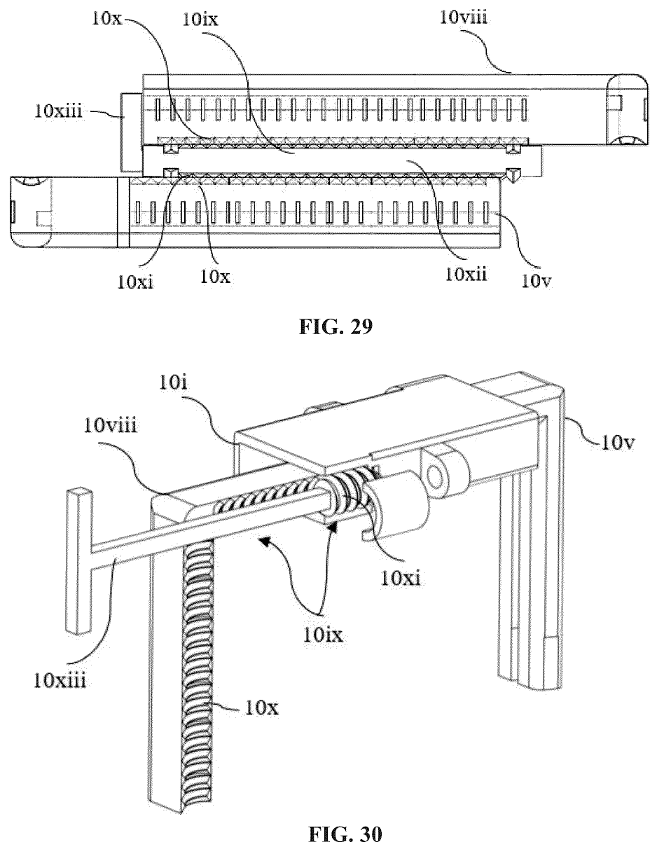

In accordance with a first embodiment, each wearable-support (10a, 10b, 10c, 10d, 10e) is provided with a first locking-configuration as shown in FIGS. 21 to 29. FIGS. 21 to 25 illustrate the housing (10i), the connecting-elements (10v, 10viii) [L-shaped elements], and the integrated locking-elements (10ix). The housing (10i) has a receptacle (10ai) to receive the connecting-elements (10v, 10viii) and the integrated locking-elements (10ix) such that the integrated locking-elements (10ix) are positioned between the connecting-elements (10v, 10viii). In one embodiment, the connecting-elements (10v, 10viii) are L-shaped elements which connect the adjacently disposed housings (10i and 10ii, 10ii and 10iii, 10iii and 10iv, and lastly 10iv and 10i) as shown in FIG. 9. FIG. 23 exemplarily illustrates the housings (10i, 10ii, 10iii, 10iv) having the receptacle (10ai) and a pivotal-connector (10aii) which is provided for connecting with the linear-adjustable-links (20a, 20b). FIG. 24 exemplarily illustrates the L-shaped elements/connecting-elements (10v, 10vi, 10vii, 10viii) having teeth (10x) configured on side-edges. FIG. 25 illustrates the integrated locking-elements (10ix) having teeth (10xi) configured on oppositely disposed circular-quarters/circular-segments, while toothless/smooth surface (10xii) on oppositely disposed circular-quarters/circular-segments. The integrated locking-elements (10ix) are manually operated through an integrated-key (10xiii) which has one-quarter turning degree of freedom. The integrated locking-elements (10ix) are positioned between the L-shaped elements/connecting-elements (10v, 10viii). FIGS. 26 and 27 illustrate the unlocking-configuration in which the integrated-key (10xiii) is manually turned to one-fourth quarter in a first-direction (as non-limitingly illustrated upward direction), the toothless/smooth surface (10xii) faces the teeth (10x) of the L-shaped elements/connecting-elements (10v, 10viii). FIGS. 28 and 29 illustrate the locking-configuration in which the integrated-key (10xiii) is manually turned to one-fourth quarter in a second-direction (as non-limitingly illustrated downward direction), the teeth (10xi) of the integrated locking-elements (10ix) engage to lock with the teeth (10x) of the L-shaped elements/connecting-elements (10v, 10viii). The first locking-configuration is preferred owing to ease of operation and the integrated-key (10xiii), which removes dependency on non-integrated elements.

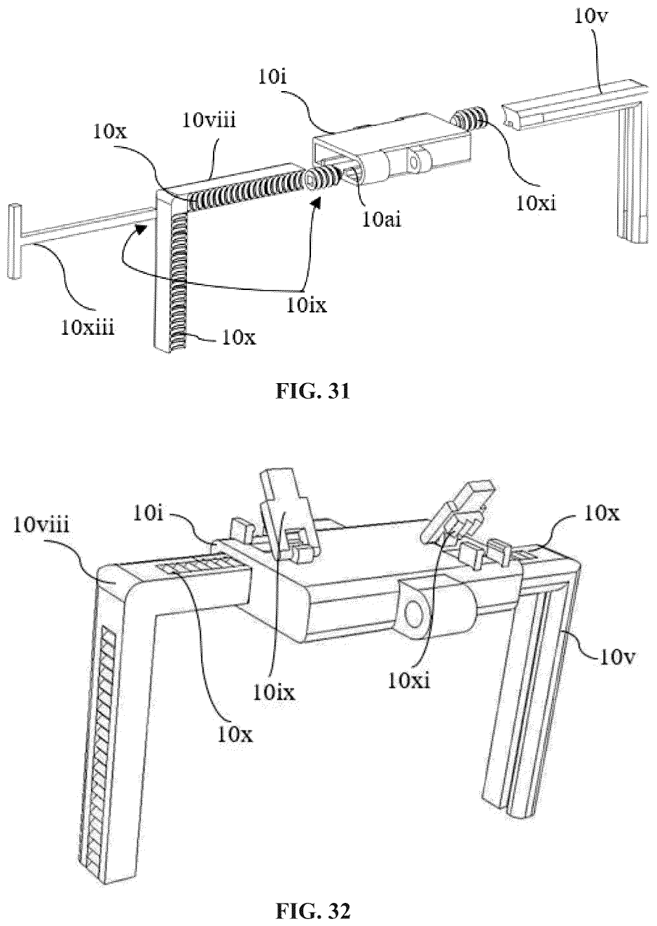

In accordance with a second embodiment, each wearable-support (10a, 10b, 10c, 10d, 10e) is provided with a second locking-configuration as shown in FIGS. 30 and 31. FIGS. 30 and 31 illustrate the housing (10i), the connecting-elements (10v, 10viii) [L-shaped elements], and the removably-connected locking-elements (10ix). Each housing (10i) has a receptacle (10ai) to receive the connecting-elements (10v, 10viii) having threads (10x) and the removably-connected locking-elements (10ix) such that the removably-connected locking-elements (10ix) are positioned for each of the connecting-elements (10v, 10viii). The removably-connected locking-elements (10ix) include a threaded-rod (10xi) and a removable-key (10xiii). The threaded-rod (10xi) is fixedly connected in the housing (10i). The removable-key (10xiii) is connected to the threaded-rod (10xi) and manually rotated in a first direction for extending the connecting-elements (10v, 10viii) and manually rotated in a second direction for retracting the connecting-elements (10v, 10viii).

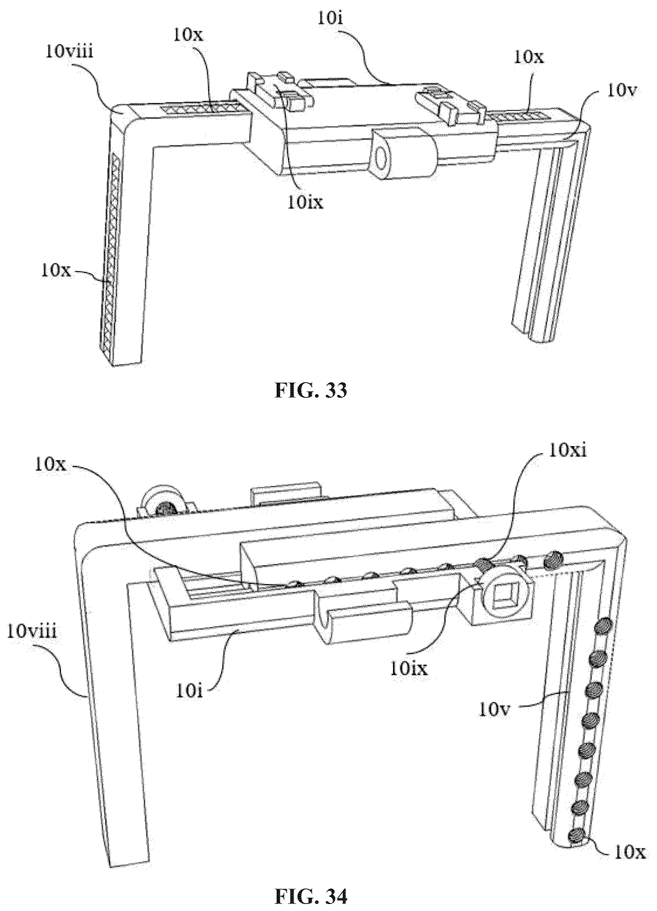

In accordance with a third embodiment, each wearable-support (10a, 10b, 10c, 10d, 10e) is provided with a third locking-configuration as shown in FIGS. 32 and 33. FIGS. 32 and 33 illustrate the housing (10i), the connecting-elements (10v, 10viii) [L-shaped elements], and the housing-integrated locking-elements (10ix). The housing-integrated locking-elements (10ix) are pivotally connected to the housing (10i) to connect and disconnect with the connecting-elements (10v, 10viii) which are movably disposed within the housing (10i). More specifically, as shown in FIG. 32, the housing-integrated locking-elements (10ix) having teeth (10xi) are pivotally lifted to unlock with the teeth (10x) of the connecting-elements (10v, 10viii). In the unlocking-configuration, the connecting-elements (10v, 10viii) are moveable (extended-out or inserted-inside) within the housing (10i) for adjustment. As shown in FIG. 33, the housing-integrated locking-elements (10ix) having teeth (10xi) are pivotally lowered to establish a connection of the teeth (10xi) with the teeth (10x) of the connecting-elements (10v, 10viii), enabling locking. In the locking-configuration, the connecting-elements (10v, 10viii) are not-moveable and are fixedly positioned within the housing (10i).

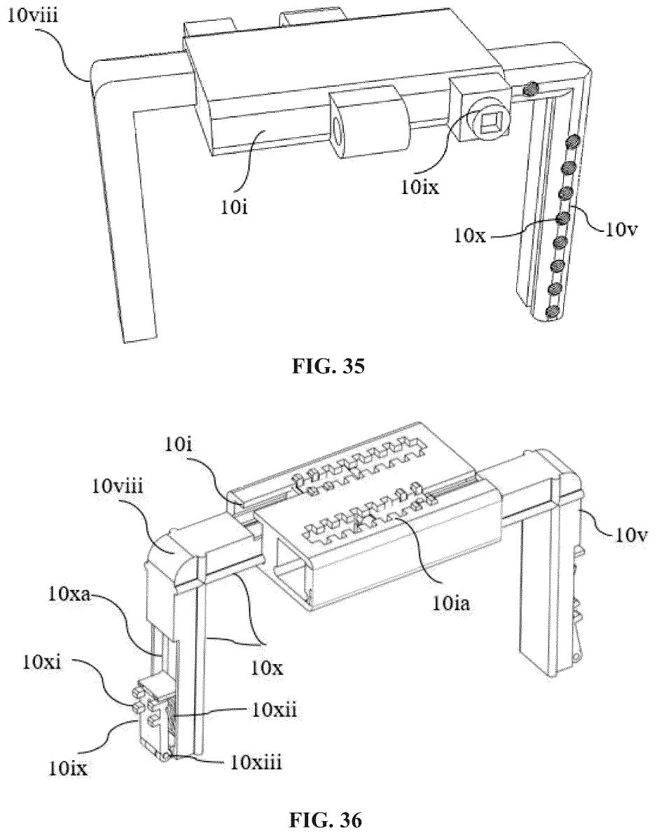

In accordance with a fourth embodiment, each wearable-support (10a, 10b, 10c, 10d, 10e) is provided with a fourth locking-configuration as shown in FIGS. 34 and 35. FIGS. 34 and 35 illustrate the housing (10i), the connecting-elements (10v, 10viii) [L-shaped elements] with engaging threaded-holes (10x), and bolt locking-elements (10ix) having threads (10xi). The bolt locking-elements (10ix) are manually rotated to engage with the threaded-holes (10x) to achieve the locking-configuration and restrict movement of the connecting-elements (10v, 10viii) in the housing (10i). The bolt locking-elements (10ix) are manually disengaged with the threaded-holes (10x) to achieve the unlocking-configuration and enable the movement (extend-out and insert-in) of the connecting-elements (10v, 10viii) in the housing (10i).

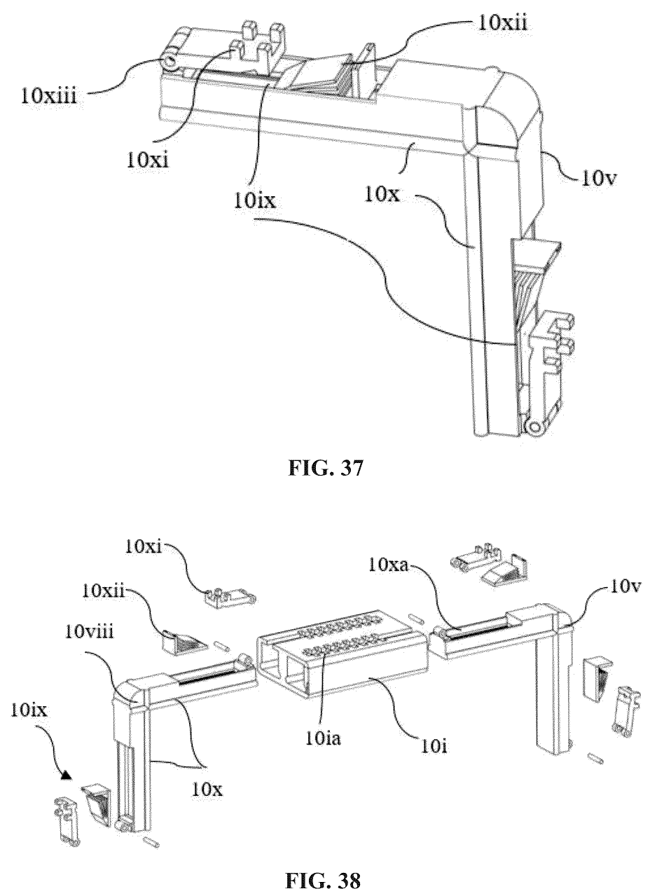

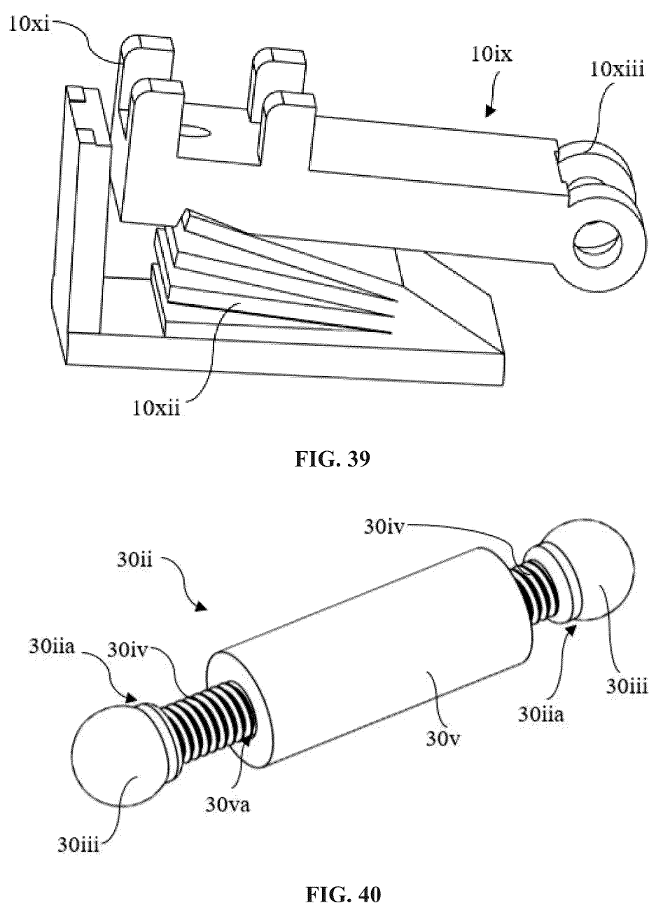

In accordance with a fifth embodiment, each wearable-support (10a, 10b, 10c, 10d, 10e) is provided with a fifth locking-configuration as shown in FIGS. 36 to 39. FIGS. 36 to 39 illustrate the housing (10i) with openings (10ia), the connecting-elements (10v, 10viii) [L-shaped elements] with a slide-rail (10x) and slidable locking-elements (10ix) having a pusher (10xi) and a retracting-element (10xii). The pusher (10xi) engages with the openings (10ia) to lock the sliding movement of the connecting-elements (10v, 10viii) in the housing (10i). The retracting-element (10xii) is movable in the slot (10xa) of the slide-rail (10x) to engage and disengage with the pusher (10xi). The pusher (10xi) is pivotally connected to the connecting-elements (10v, 10viii) and can rotate through a hinge arrangement (10xiii) as illustrated in FIG. 37. In FIG. 39, the retracting-element (10xii) is pushed to be inserted into the pusher (10xi), which causes the teeth of the pusher (10xi) to lift upward and engage with the openings (10ia), thereby achieving interlocking. Since the pusher (10xi) is hinged, gravity keeps the teeth downwardly oriented. In the event when the retracting-element (10xii) is outwardly pushed in the pusher (10xi), the teeth of the pusher (10xi) are lifted to align with the openings (10ia), enabling engagement. The retracting element (10xii) ensures that the pusher (10xi) remains in contact with the housing (10i) during interlocking.

FIG. 40 illustrates threaded-rods (30ii) of the rotational-adjustable-joints (30a), in accordance with one embodiment. The threaded-rods (30ii) are defined with threaded-portions (30iv) which are threadably fitted inside an enclosure (30v). The manual rotation of the enclosure (30v) enables to extend and retract the threaded-rods (30ii). In one embodiment, each threaded-rod (30ii) has one-end (30iia) which is defined with a ball (30iii) which is fitted/disposed in the socket (30i) [disposed on the wearable-supports (10a, 10b) as shown in FIGS. 11,12,13,15,16] and a threaded-portion (30iv) threadably fitted in an enclosure (30v) having internal thread-receptacles (30va). The manual rotation of the enclosure (30v) extends (when rotated in a first direction) and retracts (when rotated in a second direction, which is opposite to the first direction) the threaded-rods (30ii). The balls (30iii) pivotally-rotate in the sockets (30i), thereby providing the angular adjustment. In an alternative embodiment, a known ball-socket-joint can be used.

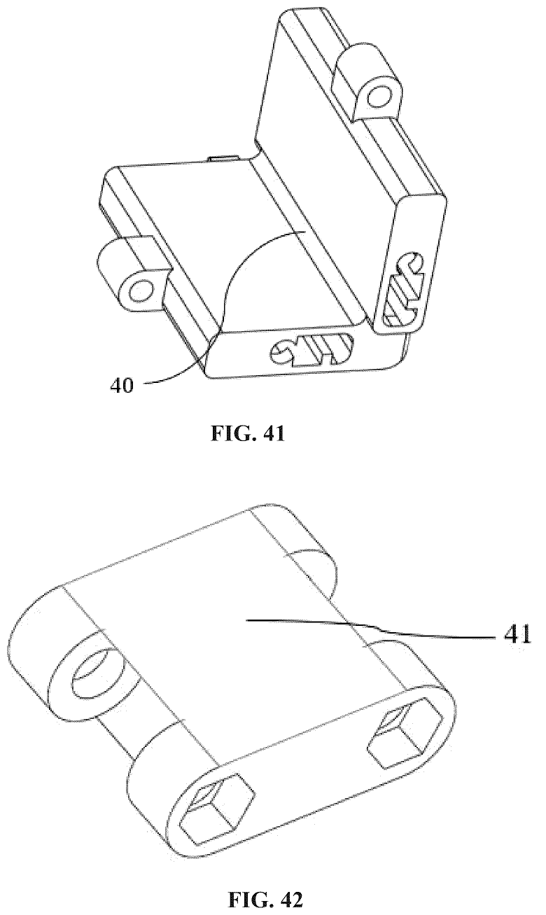

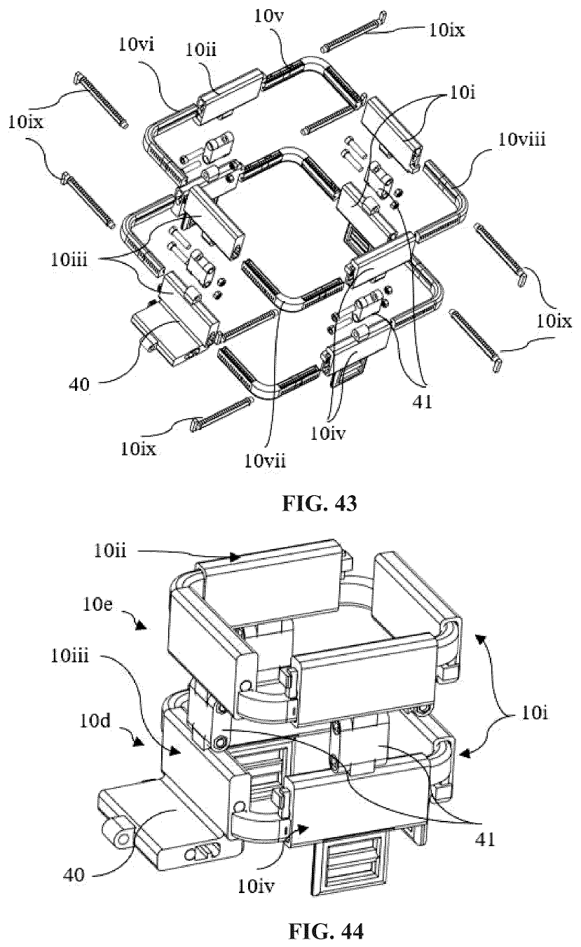

In an additional embodiment, when the limbs (05d, 05e) of the upper arm require immobilization and stabilization, an angular-connector (40), as illustrated in FIG. 41, is connected to the wearable-support (10c) to connect with the wearable-support (10d). The spaced-connectors (41), as shown in FIG. 42, are provided to establish a connection between the wearable-support (10d) and the wearable-support (10e). FIG. 43 discloses an exploded view of the wearable-support (10d) and the wearable-support (10e), the spaced-connectors (41) and the angular-connector (40) connected with the wearable-support (10d). FIG. 44 discloses an assembled view of the wearable-support (10d) and the wearable-support (10e), the spaced-connectors (41), and the angular-connector (40). The wearable-supports (10d, 10e) are structurally similar to the wearable-supports (10a, 10b, 10c). The wearable-supports (10d, 10e) include the housings (10i, 10ii, 10iii, 10iv), the connecting-elements (10v, 10vi, 10vii, 10viii), and the locking-elements (10ix). For wearing the wearable-supports (10d, 10e) on the limbs (05d, 05e), the housing (10i) and the connecting-elements (10v, 10viii) of the wearable-supports (10d, 10e) are disengaged by the locking-elements (10ix) and removed from the housings (10ii, 10iv) and the connecting-elements (10vi, 10vii), which are connected to the wearable-support (10c) through the angular-connector (40). The limbs (05d, 05e) are inserted in the housings (10ii, 10iii, 10iv) and the connecting-elements (10vi, 10vii) and then the housing (10i) and the connecting-elements (10v, 10viii) are inserted in the housings (10ii, 10iv) thus trapping/enclosing the limbs (05d, 05e) and the connecting-elements (10v, 10vi, 10vii, 10viii) are adjusted for enabling the gripping-configuration.

The detailed adjustment method and working of the adjustable-device (100) is described in subsequent paragraphs.

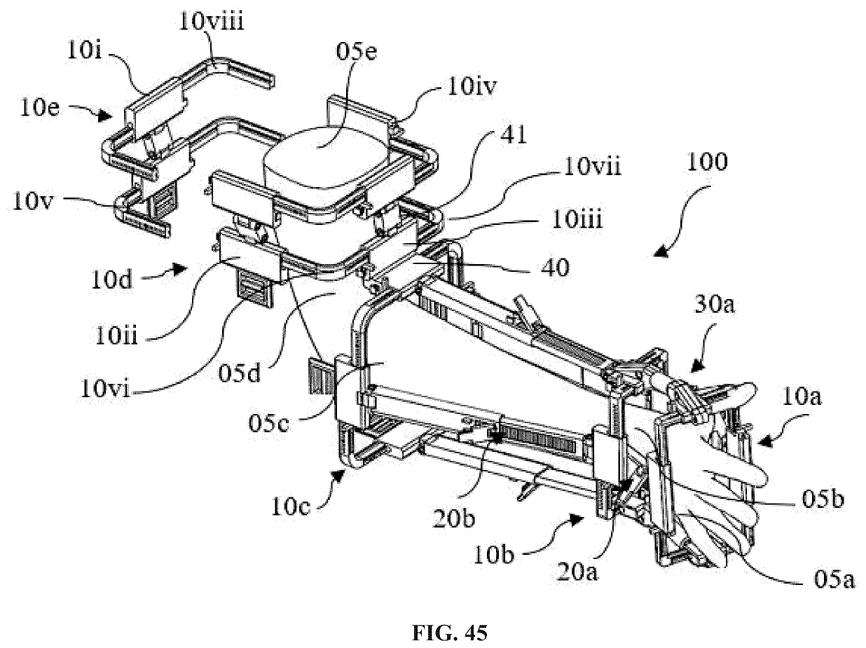

The present disclosure also discloses a method to adjust the adjustable-device (100) for immobilizing and stabilizing limbs (05a, 05b, 05c, 05d, 05e), in accordance with one embodiment of the present disclosure. FIGS. 45 to 51 depict the method to adjust the adjustable-device (100). The best-method initiates by providing the adjustable-device (100) as disclosed in the foregoing paragraphs. The adjustable-device (100) is defined with the wearable-supports (10a, 10b, 10c, 10d, 10e), the linear-adjustable-links (20a, 20b), and the rotational-adjustable-joints (30a). The linear-adjustable-links (20a, 20b) are connected (pivotally and/or fixedly) to the wearable-supports (10a, 10b, 10c), the rotational-adjustable-joints (30a) are pivotally-connected to the wearable-supports (10a, 10b). The detailed features are not described herein again to avoid repetition of the above paragraphs.

In the event when the wearable-supports (10a, 10b, 10c, 10d, 10e) are not in the expanded-configuration, the wearable-supports (10a, 10b, 10c, 10d, 10e) are adjusted to the expanded-configuration as illustrated in FIG. 45. The expanded-configuration is achieved by unlocking the locking-elements (10ix) to disengage the connecting-elements (10v, 10vi, 10vii, 10viii) with the housings (10i, 10ii, 10iii, 10iv) to enable outward-movement of the connecting-elements (10v, 10vi, 10vii, 10viii) in the housings (10i, 10ii, 10iii, 10iv) for expansion of the wearable-supports (10a, 10b, 10c, 10d, 10e). Further, for wearing the wearable-supports (10d, 10e) on the limbs (05d, 05e), the connecting-elements (10v, 10viii) along with the housing (10i) of the wearable-supports (10d, 10e) are pulled-out to disengage from the housings (10ii, 10iv). More specifically, upon disengaging the connecting-elements (10v, 10viii) along with the housing (10i) from the housings (10ii, 10iv), the connecting-elements (10v, 10viii) along with the housings (10i) are extended-out to be detached from the housings (10ii, 10iv). Upon extending-out, the limbs (05a, 05b, 05c, 05d, 05e) are inserted in the wearable-supports (10a, 10b, 10c, 10d, 10e) as shown in FIGS. 45, 46, and 47.

While or before inserting the limbs (05a, 05b, 05c), the linear-adjustable-links (20a, 20b) are unlocked and are actuated to linearly-adjust the distance between the wearable-supports (10a, 10b, 10c) to position the wearable-supports (10a, 10b, 10c) on the limbs (05a, 05b, 05c) and then locked. As disclosed, each of the linear-adjustable-links (20a, 20b) is defined with the female-element (20i), the male-element (20ii) with male-interlocking-teeth (20iii), and the locking-member (20iv) which is defined on the female-element (20i). For unlocking, the locking-member (20iv) is operated to allow telescopic-adjustment of the male-element (20ii) in or out of the female-element (20i), and once the length is adjusted, the locking-member (20iv) is locked to engage with the male-interlocking-teeth (20iii) and arrest the male-element (20ii) with the female-element (20i).

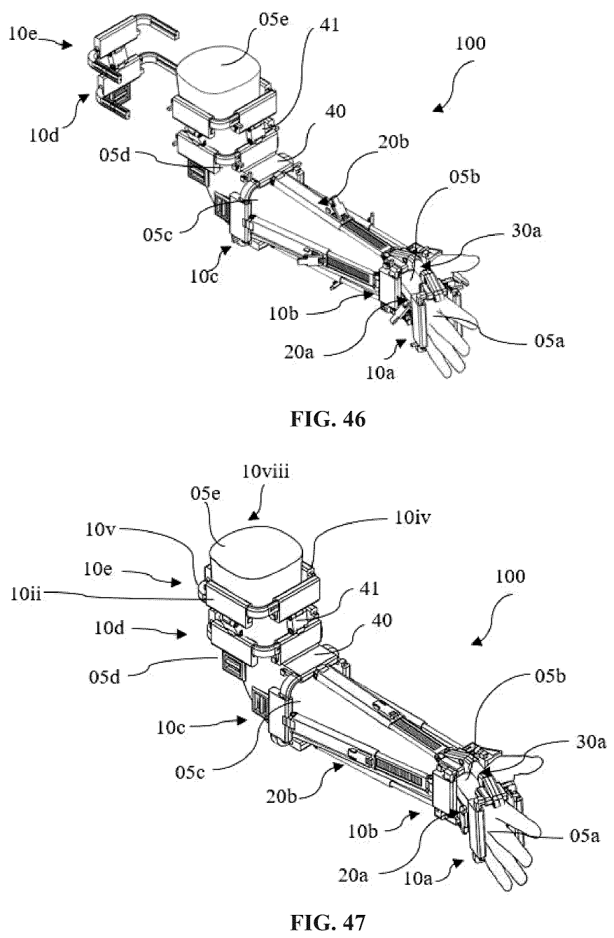

The wearable-supports (10a, 10b, 10c) are adjusted by compressing the wearable-supports (10a, 10b, 10c) to grip with the limbs (05a, 05b, 05c) by inwardly moving the connecting-elements (10v, 10vi, 10vii, 10viii) in the housings (10i, 10ii, 10iii, 10iv), followed by locking of the locking-elements (10ix) to engage the connecting-elements (10v, 10vi, 10vii, 10viii) with the housings (10i, 10ii, 10iii, 10iv) to achieve the gripping-configuration with the limbs (05a, 05b, 05c) as shown in FIGS. 46, 47, 49 and 50. Further, in one embodiment, the wearable-supports (10d, 10e) achieve the gripping-configuration by inserting the previously detached connecting-elements (10v, 10viii) along with the housing (10i) in the housings (10ii, 10iv). Once the connecting-elements (10v, 10viii) along with the housing (10i) are inserted in the housings (10ii, 10iv), the connecting-elements (10v, 10vi, 10vii, 10viii) of the wearable-supports (10d, 10e) are compressed to grip with the limbs (05d, 05e) and the locking-elements (10ix) engage the connecting-elements (10v, 10vi, 10vii, 10viii) with the housings (10i, 10ii, 10iii, 10iv) for achieving the gripping-configuration.

The rotational-adjustable-joints (30a) are also adjusted for rotationally-adjusting the dimensional-variance of the adjacently-disposed wearable-supports (10a, 10b). The dimensional-variance is the difference-in-sizes of the adjacently-disposed wearable-supports (10a, 10b) worn on the limbs (05a, 05b) after compression. In one embodiment, adjusting the rotational-adjustable-joints (30a), which are defined with the sockets (30i) defined on adjacently-disposed wearable-supports (10a, 10b), the threaded-rods (30ii) with the balls (30iii) and the threaded-portions (30iv), and the enclosure (30v) having internal thread-receptacles (30va), includes rotating the enclosure (30v), for extending and retracting the threaded-rods (30ii) to be equivalent to the extended or retracted length of the adjusted linear-adjustable-links (20a). The balls (30iii) as are freely-disposed in the sockets (30i) are pivotally-rotated in the sockets (30i), thus enabling rotational-adjustments.

In the event when the limbs (05d, 05e) of the upper arm need immobilization and stabilization, the adjustable-device (100) as shown in FIG. 1 is provided. The wearable-supports (10a, 10b, 10c, 10d, 10e) are unlocked and expanded into the expanded-configuration. Initially, the wearable-supports (10d, 10e) are partially removed as illustrated in FIG. 45. The limbs (05a, 05b, 05c) are inserted in the wearable-supports (10a, 10b, 10c). The linear-adjustable-links (20a, 20b) are unlocked and are actuated to linearly-adjust the distance between the wearable-supports (10a, 10b, 10c) to position the wearable-supports (10a, 10b, 10c) on the limbs (05a, 05b, 05c) and are locked. The wearable-supports (10a, 10b, 10c) are manually compressed to achieve the gripping-configuration as shown in FIG. 46. For wearing the wearable-supports (10d, 10e) on the limbs (05d, 05e), the housing (10i) and the connecting-elements (10v, 10viii) of the wearable-supports (10d, 10e) are removed from the housings (10ii, 10iv) and the connecting-elements (10vi, 10vii) which are connected to the wearable-support (10c) through the angular-connector (40). The limbs (05d, 05e) are inserted in the housings (10ii, 10iii, 10iv) which are connected by the connecting-elements (10vi, 10vii) and then the housing (10i) and the connecting-elements (10v, 10viii) are inserted in the housings (10ii, 10iv) and the connecting-elements (10vi, 10vii) (as shown in FIG. 47) thus trapping/enclosing the limbs (05d, 05e) and the connecting-elements (10v, 10vi, 10vii, 10viii) are adjusted to enable the gripping-configuration. The rotational-adjustable-joints (30a) are adjusted for rotationally-adjusting the dimensional-variance of the adjacently-disposed wearable-supports (10a, 10b).

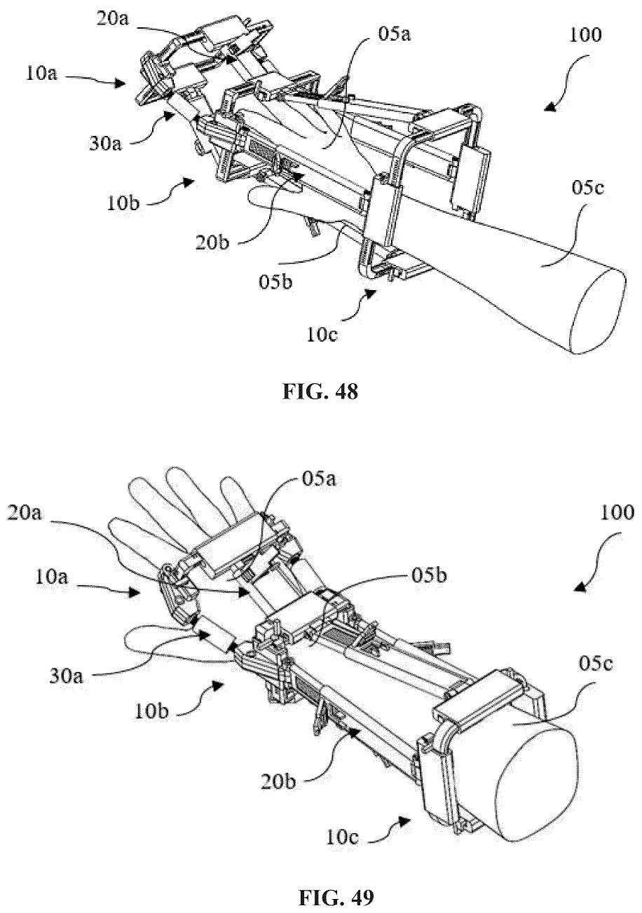

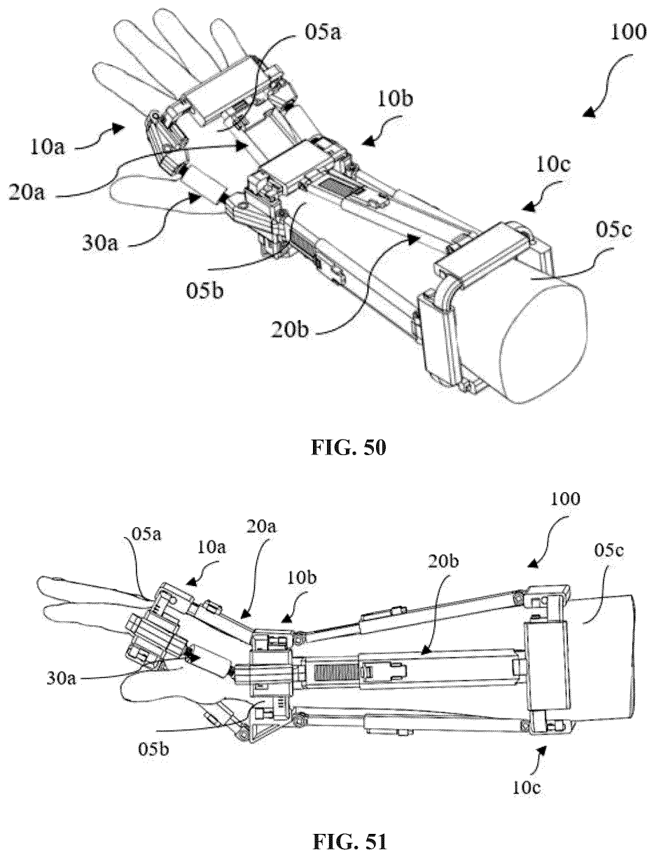

In the event if only the limbs (05a, 05b, 05c) need immobilization and stabilization, the wearable-supports (10a, 10b, 10c) are unlocked and expanded into the expanded-configuration, and the limbs (05a, 05b, 05c) are inserted as shown in FIG. 48. The linear-adjustable-links (20a, 20b) are unlocked and are actuated to linearly-adjust the distance between the wearable-supports (10a, 10b, 10c) to position the wearable-supports (10a, 10b, 10c) on the limbs (05a, 05b, 05c) as shown in FIG. 49. The linear-adjustable-links (20a, 20b) are then locked as shown in FIG. 50. The wearable-supports (10a, 10b, 10c) are compressed and locked into the gripping-configuration with the limbs (05a, 05b, 05c). The rotational-adjustable-joints (30a) are adjusted for rotationally-adjusting the dimensional-variance of the adjacently-disposed wearable-supports (10a, 10b). FIG. 51 shows the immobilized and stabilized limbs (05a, 05b, 05c) by adjusting the adjustable-device (100).

In accordance with a first embodiment, each wearable-support (10a, 10b, 10c, 10d, 10e) is provided with a first locking-configuration as shown in FIGS. 21 to 29. FIGS. 21 to 25 illustrate the housing (10i), the connecting-elements (10v, 10viii) [L-shaped elements], and the integrated locking-elements (10ix). The housing (10i) has a receptacle (10ai) to receive the connecting-elements (10v, 10viii) and the integrated locking-elements (10ix) such that the integrated locking-elements (10ix) are positioned between the connecting-elements (10v, 10viii). In one embodiment, the connecting-elements (10v, 10viii) are L-shaped elements that connect adjacently disposed housings (10i and 10ii, 10ii and 10iii, 10iii and 10iv, and lastly 10iv and 10i) as shown in FIG. 9. FIG. 23 exemplarily illustrates the housings (10i, 10ii, 10iii, 10iv) having the receptacle (10ai) and a pivotal-connector (10aii) which is used for connecting with the linear-adjustable-links (20a, 20b). FIG. 24 exemplarily illustrates the L-shaped elements/connecting-element (10v, 10vi, 10vii, 10viii) having teeth (10x) configured on side-edges. FIG. 25 illustrates the integrated locking-elements (10ix) having teeth (10xi) configured on oppositely disposed circular-quarters/circular-segments, while the toothless/smooth surface (10xii) on oppositely disposed circular-quarters/circular-segments. The integrated locking-elements (10ix) are manually operated through an integrated-key (10xiii) which has one-quarter turning degree of freedom. The integrated locking-elements (10ix) are positioned between the L-shaped elements/connecting-elements (10v, 10viii). FIGS. 26 and 27 illustrate the unlocking-configuration in which the integrated-key (10xiii) is manually turned to one-fourth quarter in a first-direction (as non-limitingly illustrated upward direction), the toothless/smooth surface (10xii) faces the teeth (10x) of the L-shaped elements/connecting-elements (10v, 10viii). FIGS. 28 and 29 illustrate the locking-configuration in which the integrated-key (10xiii) is manually turned to one-fourth quarter in a second-direction (as non-limitingly illustrated downward direction), the teeth (10xi) of the integrated locking-elements (10ix) engage to lock with the teeth (10x) of the L-shaped elements/connecting-elements (10v, 10viii). The first locking configuration is preferred owing to the ease of operation and the integrated-key (10xiii) which removes dependency on non-integrated elements.

In accordance with a second embodiment, each wearable-support (10a, 10b, 10c, 10d, 10e) is provided with a second locking-configuration as shown in FIGS. 30 and 31. FIGS. 30 and 31 illustrate the housing (10i), the connecting-elements (10v, 10viii) [L-shaped elements], and the removably-connected locking-elements (10ix). The housing (10i) has a receptacle (10ai) to receive the connecting-elements (10v, 10viii) having threads (10x) and the removably-connected locking-elements (10ix) such that the removably-connected locking-elements (10ix) are positioned for each of the connecting-elements (10v, 10viii). The removably-connected locking-elements (10ix) include a threaded-rod (10xi) and a removable-key (10xiii). The threaded-rod (10xi) is fixedly connected in the housing (10i). The removable-key (10xiii) is connected to the threaded rod (10xi) and manually rotated in a first direction for extending the connecting-elements (10v, 10viii) and manually rotated in a second direction for retracting the connecting-elements (10v, 10viii).

In accordance with a third embodiment, each wearable-support (10a, 10b, 10c, 10d, 10e) is provided with a third locking-configuration as shown in FIGS. 32 and 33. FIGS. 32 and 33 illustrate the housing (10i), the connecting-elements (10v, 10viii) [L-shaped elements], and the housing-integrated locking-elements (10ix). The housing-integrated locking-elements (10ix) are pivotally connected to the housing (10i) to connect and disconnect with the connecting-elements (10v, 10viii) movably disposed within the housing (10i). More specifically, as shown in FIG. 32, the housing-integrated locking-elements (10ix) having teeth (10xi) are pivotally lifted to unlock from the teeth (10x) of the connecting-elements (10v, 10viii). In the unlocking-configuration, the connecting-elements (10v, 10viii) are moveable (extended-out or inserted-inside) within the housing (10i) for adjustment. As shown in FIG. 33, the housing-integrated locking-elements (10ix) having the teeth (10xi) are pivotally lowered to establish a connection of the teeth (10xi) with the teeth (10x) of the connecting-elements (10v, 10viii), enabling locking. In the locking-configuration, the connecting-elements (10v, 10viii) are not-moveable and are fixedly positioned within the housing (10i).

In accordance with a fourth embodiment, each wearable-support (10a, 10b, 10c, 10d, 10e) is provided with a fourth locking-configuration as shown in FIGS. 34 and 35. FIGS. 34 and 35 illustrate the housing (10i), the connecting-elements (10v, 10viii) [L-shaped elements] with engaging threaded-holes (10x), and bolt locking-elements (10ix) having threads (10xi). The bolt locking-elements (10ix) are manually rotated to engage with the threaded-holes (10x) to achieve the locking-configuration and restrict the movement of the connecting-elements (10v, 10viii) in the housing (10i). The bolt locking-elements (10ix) are manually disengaged from the threaded-holes (10x) to achieve the unlocking-configuration and enable movement (extend-out and insert-in) of the connecting-elements (10v, 10viii) in the housing (10i).

In accordance with a fifth embodiment, each wearable-support (10a, 10b, 10c, 10d, 10e) is provided with a fifth locking-configuration as shown in FIGS. 36 and 39. FIGS. 36 and 39 illustrate the housing (10i) with openings (10ia), the connecting-elements (10v, 10viii) [L-shaped elements] with a slide-rail (10x) and slidable locking-elements (10ix) having a pusher (10xi) and a retracting-element (10xii). The pusher (10xi) engages with the openings (10ia) to lock the sliding movement of the connecting-elements (10v, 10viii) in the housing (10i). The retracting-element (10xii), is movable within the slot (10xa) of the slide-rail (10x) to engage and disengage with the pusher (10xi). The pusher (10xi) is pivotally connected to the connecting-elements (10v, 10viii) and can rotate through a hinge arrangement (10xiii) as illustrated in FIG. 37. In FIG. 39, the retracting-element (10xii) is pushed to be inserted into the pusher (10xi) which causes the teeth of the pusher (10xi) to lift upward and engage with the openings (10ia), thereby achieving interlocking. Since the pusher (10xi) is hinged, gravity keeps the teeth downwardly oriented. In the event when the retracting-element (10xii) is outwardly pushed into the pusher (10xi), the teeth of the pusher (10xi) are lifted to align with the openings (10ia), enabling engagement. The retracting element (10xii) ensures that the pusher (10xi) remains in contact with the housing (10i) during interlocking.

In an additional embodiment, as illustrated in FIGS. 52 to 55, the linear-adjustable-links (20a) are angularly adjusted (manually by rotating a known allen-key) by the angular-adjustment-mechanism (20c) for angularly adjusting the linear-adjustable-links (20a). The angular-adjustment-mechanism (20c) is defined with a left-right-adjustment and a top-bottom adjustment. The left-right adjustment is movable in direction X, and the top-bottom adjustment is movable in direction Y, as illustrated in FIG. 52. The left-right-adjustment includes a worm-wheel (20ci) with teeth (20cii) and a teethed-worm (20ciii) with teeth (20civ). The teethed-worm (20ciii) is rotated to rotate the worm-wheel (20ci), enabling left and right movement depending upon clockwise and anticlockwise rotation of the teethed-worm (20ciii). The worm-wheel (20ci) is defined on the male-element (20ii). The teethed-worm (20ciii) is rotatably disposed in an extended-portion (10bi) of the wearable-support (10b). The teeth (20civ) are connected to the teeth (20cii). The top-bottom-adjustment includes a worm (20cv) with teeth (20cvi) and an angular-teethed-wheel (20cvii) with angular-teeth (20cviii). The worm (20cv) is disposed in the extended-portion (10bi) and having axis transverse to the axis of the teethed-worm (20ciii). The angular-teethed-wheel (20cvii) extends to connect with the worm-wheel (20ci) by use of a fastener (20cix). The teeth (20cvi) are connected to the angular-teeth (20cviii). The worm (20cv) is rotated to rotate the angular-teethed-wheel (20cvii) to achieve top-bottom adjustment (Y). In an additional embodiment, as illustrated in FIGS. 54 and 55, the linear-adjustable-link (20ai) is provided with a freewheeling left-right movements, and the left-right movement of the linear-adjustable-link (20ai) is in accordance with the left-right adjustment of the linear-adjustable-link (20a). The linear-adjustable-link (20ai) differs from the linear-adjustable-link (20a) by using a toothless-wheel (20d) pivotally movable (in the left direction ‘L’ and in the right direction ‘R’) and is pivotally-connected by a fastener (20cix).

The adjustable-device (100) and method to adjust the adjustable-device (100) for immobilizing and stabilizing limbs (05a, 05b, 05c, 05d, 05e) is fully adjustable, which includes linear adjustment and rotational adjustment, before and after application of the adjustable-device (100) on limbs (05a, 05b, 05c, 05d, 05e), allowing it to adapt to different limb sizes and fluctuations due to swelling or its reduction, facilitate dressing changes, enhance patient comfort and stability throughout the healing process, and provide air ventilation for hygiene while being waterproof, washable, and designed to minimize discomfort. The adjustment can be done by the user (05) wearing the adjustable-device (100) or the adjustment can be performed by any person who assists the user (05). The major advantage of the adjustable-device (100) is linear and rotational adjustment, with adjustment performed pre- or post-application, for example, before applying the adjustable-device (100) and adjusting after witnessing swollen limbs or reduction in swelling, which is conventionally not disclosed. The adjustable-device (100) and method to adjust the adjustable-device (100) for immobilizing and stabilizing limbs (05a, 05b, 05c, 05d, 05e) eliminates the need for traditional hardening agents like water, UV light, or heat and avoids the need for associated equipment, thereby simplifying the application device and process and enabling immediate use without dependence on specialized equipment. The adjustable-device (100) and method to adjust the adjustable-device (100) for immobilizing and stabilizing limbs (05a, 05b, 05c, 50d, 05e) has air gaps that allows air circulation for better breathability and moisture control, reducing the risk of skin irritation, itching, and odor associated with traditional casting methods. The adjustable-device (100) and method to adjust the adjustable-device (100) for immobilizing and stabilizing limbs (05a, 05b, 05c, 05d, 05e) is reusable and environmentally friendly and reduces waste and lowers costs by enabling multiple uses for the same patient, and comparatively longer shelf life, and can be made from recycled materials. The adjustable-device (100) and method to adjust the adjustable-device (100) for immobilizing and stabilizing limbs (05a, 05b, 05c, 05d, 05e) are adjustable to accommodate a broad range of limb sizes and anatomies without requiring different cast sizes, making it a more universally applicable and convenient option. In additional embodiment, the adjustable-device (100) can be internally provided with a layer of padding (not shown) that provides a cushioning effect. The adjustable-device (100) can be made of polymers, metals, ceramics, glasses, composites, natural materials, synthetic materials, or a combination thereof.

The table 1 shows the advantages of the adjustable-device (100) of the present disclosure with known prior-arts.

| | Advanced | | | | | |
|---|---|---|---|---|---|---|
| | Adjustable | | | | Resin | 3D |
| Key | Orthopedic | Plaster | | | Sleeve | Printed |
| Parameters | Cast | (POP) | Fiberglass | UV-Based | (Cast 21) | Cast |
| 1. | Yes | No | No | No | No | No |
| Adjustable | | | | | | |
| for Swelling | | | | | | |
| After | | | | | | |
| Hardening | | | | | | |
| 2. Dressing | Yes | No | No | No | No | Yes |
| Application | | | | | | |
| on Injured | | | | | | |
| Area | | | | | | |
| 3. Reapply | Yes | New cast | New cast | New cast | New cast | Yes |
| After | | is needed | is needed | is needed | is needed | |
| Dressing | | | | | | |
| Changes | | | | | | |
| 4. Average | 0.25 | 1-1.5 | 0.4-0.7 | 0.6-0.8 | 0.8-1 | 0.2-0.3 |
| Weight | kg | kg | kg | kg | kg | kg |
| (Including | | | | | | |
| Padding) | | | | | | |
| 5. | Yes | No | No | No | No | Yes |
| Prefabricated | | | | | | |
| 20° Wrist | | | | | | |
| Angle | | | | | | |
| 6. Risk of | Minimal | Might | Might | Might | Chances of | Requires |
| Application | | stick to | stick to | harden | spillage, | precise |
| (Dust, | | skin, | skin, | unevenly, | messy | scanning |
| Spillage, | | angle | angle | skin is | mixing | & fit; |
| Angle, | | often | often | exposed to | | printing |
| Exposure) | | imprecise | imprecise | UV rays | | errors |
| | | | | | | possible |
| 7. All-in- | Yes | Yes | Yes | No | No | No |
| One Size | | | | | | |
| 8. Easy | Yes | Moderate | Moderate | Moderate | Moderate | Yes |
| Application | | | | | | |
| 9. | Less than 5 | 15-30 | 10-20 | ~3 | 15-20 | Less than |
| Application | minutes | minutes | minutes | minutes | minutes | 5 minutes |
| Time | | | | | | |
| 10. Instant | Yes | No | No | No | No | Yes |
| Hardening | | | | | | |
| 11. | 0 | 24 | 30 | 1-2 | 10 seconds | 0 |
| Hardening | second | hours | minutes | hours | after | second |
| Time (Full | | | | | exposure | |
| Strength) | | | | | to UV | |
| 12. | No | Yes- | Yes- | Yes- | Yes- | No |
| Hardening | | Water | Water | Resin | UV | |
| Agent or | | | | | Light | |
| Equipment | | | | | Source | |
| Required | | | | | | |
| 13. Easy | Yes | No | No | No | No | Yes |
| Removal | | | | | | |
| (No Cutting | | | | | | |
| Equipment) | | | | | | |
| 14. | Yes | No | No | Yes | Yes | Yes |
| Improved | | | | | | |
| Hygiene | | | | | | |
| 15. | Yes | No | No | Yes | Yes | Yes |
| Waterproof | | | | | | |
| 16. | Yes | No | No | Yes | Yes | Yes |
| Breathable | | | | | | |
| 17. Odor | Yes | No | No | Yes | Yes | Yes |
| Resistant | | | | | | |
| 18. Easy to | Yes | No | No | Yes | Yes | Yes |
| Clean | | | | | | |
| 19. | No | Yes | Yes | Yes | Yes | No |
| Chemicals | | | | | | |
| Used | | | | | | |
| 20. | Yes | No | No | No | No | Yes |
| Unlimited | | | | | | |
| Shelf Life | | | | | | |
| 21. Special | No | Yes | Yes | Yes | Yes | No |
| Storage | | | | | | |
| Conditions | | | | | | |
| 22. | Yes | No | No | No | No | Yes |
| Reusability | | | | | | |
| for the | | | | | | |
| Same | | | | | | |
| Patient | | | | | | |
| 23. Can be | Yes | No | No | No | No | No |
| made from | | | | | | |
| Recycled | | | | | | |
| Materials | | | | | | |

The foregoing description conveys the best understanding of the objectives and advantages of the present invention. Different embodiments, steps, or alternatives may be made of the inventive concept of this invention. It is to be understood that all matter disclosed herein is to be interpreted merely as illustrative, and not in a limiting sense.

What is claimed is:

1. An adjustable-device (100), for immobilizing and stabilizing limbs (05a, 05b, 05c, 05d, 05e), comprising:
Characterized by:
wearable-supports (10a, 10b, 10c, 10d, 10e) adjustably-wearable on said limbs (05a, 05b, 05c, 05d, 05e) and adjustable between an expanded-configuration and a gripping-configuration;
linear-adjustable-links (20a, 20b) connected to said wearable-supports (10a, 10b, 10c) and defined to linearly-adjust the distance between said wearable-supports (10a, 10b, 10c); and
rotational-adjustable-joints (30a) pivotally-connected to said wearable-supports (10a, 10b) and defined to rotationally-adjust the dimensional-variance of adjacently-disposed said wearable-supports (10a, 10b), wherein the dimensional-variance is the difference-in-sizes of adjacently-disposed said wearable-supports (10a, 10b) worn on said limbs (05a, 05b).

2. The adjustable-device (100), for immobilizing and stabilizing limbs (05a, 05b, 05c, 05d, 05e) as claimed in claim 1, wherein each of said wearable-supports (10a, 10b, 10c, 10d, 10e) is defined with housings (10i, 10ii, 10iii, 10iv), connecting-elements (10v, 10vi, 10vii, 10viii), and locking-elements (10ix), said housings (10i, 10ii, 10iii, 10iv) are connected by said connecting-elements (10v, 10vi, 10vii, 10viii) to form a wearable-structure, said connecting-elements (10v, 10vi, 10vii, 10viii) are adjustably-movable in said housings (10i, 10ii, 10iii, 10iv), said locking-elements (10ix) are defined to lock and unlock said connecting-elements (10v, 10vi, 10vii, 10viii) with said housings (10i, 10ii, 10iii, 10iv), wherein said locking-elements (10ix) locking said connecting-elements (10v, 10vi, 10vii, 10viii) with said housings (10i, 10ii, 10iii, 10iv) defining a locking-configuration and said locking-elements (10ix) un-locking said connecting-elements (10v, 10vi, 10vii, 10viii) with said housings (10i, 10ii, 10iii, 10iv) defining an un-locking-configuration.

3. The adjustable-device (100), for immobilizing and stabilizing limbs (05a, 05b, 05c, 05d, 05e) as claimed in claim 1, wherein each of said linear-adjustable-links (20a, 20b) is defined with a female-element (20i), a male-element (20ii) adjustably and telescopically movable in said female-element (20i) and is defined with male-interlocking-teeth (20iii), and a locking-member (20iv) defined on said female-element (20i) and configured to: —engage with said male-interlocking-teeth (20iii) defining a locking-configuration and —disengage with said male-interlocking-teeth (20iii) defining an un-locking-configuration.

4. The adjustable-device (100), for immobilizing and stabilizing limbs (05a, 05b, 05c, 05d, 05e) as claimed in claim 1, wherein each of said rotational-adjustable-joints (30a) is defined with sockets (30i) defined on adjacently-disposed said wearable-supports (10a, 10b), threaded-rods (30ii), each said threaded-rod (30ii) having one-end (30iia) defined with a ball (30iii) fitted in said socket (30i) and a threaded-portion (30iv) threadably fitted in an enclosure (30v) having internal thread-receptacles (30va), wherein rotation of said enclosure (30v) extends and retracts said threaded-rods (30ii), said balls (30iii) pivotally-rotate in said sockets (30i).

5. The adjustable-device (100), for immobilizing and stabilizing limbs (05a, 05b, 05c, 05d, 05e) as claimed in claim 1, includes an angular-connector (40) connected between angularly-disposed said wearable-supports (10c, 10d) and a spaced-connector (41) connected between said wearable-supports (10d, 10e).

6. A method to adjust an adjustable-device (100) for immobilizing and stabilizing limbs (05a, 05b, 05c, 05d, 05e), said method comprising:
Characterized by:
providing the adjustable-device (100) defined with wearable-supports (10a, 10b, 10c, 10d, 10e), linear-adjustable-links (20a, 20b), and rotational-adjustable-joints (30a), said linear-adjustable-links (20a, 20b) connected to said wearable-supports (10a, 10b, 10c), said rotational-adjustable-joints (30a) pivotally-connected to said wearable-supports (10a, 10b);
adjusting said wearable-supports (10a, 10b, 10c, 10d, 10e) to an expanded-configuration;
inserting said limbs (05a, 05b, 05c, 05d, 05e) in said wearable-supports (10a, 10b, 10c, 10d, 10e);
adjusting linearly said linear-adjustable-links (20a, 20b) to linearly-adjust the distance between said wearable-supports (10a, 10b, 10c) to position said wearable-supports (10a, 10b, 10c) on said limbs (05a, 05b, 05c);
adjusting said wearable-supports (10a, 10b, 10c, 10d, 10e) to a gripping-configuration; and
adjusting said rotational-adjustable-joints (30a) to rotationally-adjust the dimensional-variance of adjacently-disposed said wearable-supports (10a, 10b),
wherein the dimensional-variance is the difference-in-sizes of adjacently-disposed said wearable-supports (10a, 10b) worn on said limbs (05a, 05b).

7. The method to adjust the adjustable-device (100) for immobilizing and stabilizing limbs (05a, 05b, 05c, 05d, 05e) as claimed in claim 6, wherein adjusting said wearable-supports (10a, 10b, 10c, 10d, 10e), defined with housings (10i, 10ii, 10iii, 10iv), connecting-elements (10v, 10vi, 10vii, 10viii), and locking-elements (10ix), —in the expanded-configuration includes unlocking, to achieve an unlocking-configuration, said locking-elements (10ix) to disengage said connecting-elements (10v, 10vi, 10vii, 10viii) with said housings (10i, 10ii, 10iii, 10iv) to enable outward-movement of said connecting-elements (10v, 10vi, 10vii, 10viii) in said housings (10i, 10ii, 10iii, 10iv) for expansion of said wearable-supports (10a, 10b, 10c, 10d, 10e) for inserting said limbs (05a, 05b, 05c, 05d, 05e); and
in the gripping-configuration by inwardly moving said connecting-elements (10v, 10vi, 10vii, 10viii) in said housings (10i, 10ii, 10iii, 10iv) to grip with said limbs (05a, 05b, 05c, 05d, 05e) and locking, to achieve a locking-configuration, said locking-elements (10ix) to engage said connecting-elements (10v, 10vi, 10vii, 10viii) with said housings (10i, 10ii, 10iii, 10iv) for the gripping-configuration.

8. The method to adjust the adjustable-device (100) for immobilizing and stabilizing limbs (05a, 05b, 05c, 05d, 05e) as claimed in claim 7, wherein disengaging and reengaging said connecting-elements (10v, 10viii), connected to said housing (10i) of said wearable-supports (10d, 10e), from said housings (10ii, 10iii, 10iv) assembled with said connecting-elements (10vi, 10vii) of said wearable-supports (10d, 10e), specifically for inserting, and immobilizing and stabilizing said limbs (05d, 05e).

9. The method to adjust the adjustable-device (100) for immobilizing and stabilizing limbs (05a, 05b, 05c, 05d, 05e) as claimed in claim 6, adjusting linearly said linear-adjustable-links (20a, 20b), defined with a female-element (20i), a male-element (20ii) with male-interlocking-teeth (20iii), and a locking-member (20iv) defined on said female-element (20i), includes unlocking said locking-member (20iv) to telescopically-adjust said male-element (20ii) in said female-element (20i) and locking said locking-member (20iv) to engage with said male-interlocking-teeth (20iii) and arrest said male-element (20ii) with said female-element (20i).

10. The method to adjust the adjustable-device (100) for immobilizing and stabilizing limbs (05a, 05b, 05c, 05d, 05e) as claimed in claim 6, wherein adjusting said rotational-adjustable-joints (30a), defined with sockets (30i) defined on adjacently-disposed said wearable-supports (10a, 10b), threaded-rods (30ii) with balls (30iii) and threaded-portions (30iv), and an enclosure (30v) having internal thread-receptacles (30va), includes rotating said enclosure (30v) for extending and retracting said threaded-rods (30ii), said balls (30iii) pivotally-rotate in said sockets (30i).
